SEQUENCE LISTING (6 sequences)

SEQ ID NO: 1 - DNA, 20 bp, synthetic construct
  source 1..20: mol_type = other DNA
aacccaagcacgtatcaggg

SEQ ID NO: 2 - DNA, 20 bp, synthetic construct
  source 1..20: mol_type = other DNA
actgctggtcaggatcgttg

SEQ ID NO: 3 - DNA, 19 bp, synthetic construct
  source 1..19: mol_type = other DNA
aatgtgttcagtgcagagc

SEQ ID NO: 4 - DNA, 18 bp, synthetic construct
  source 1..18: mol_type = other DNA
ttgggatgatgtcgggac

SEQ ID NO: 5 - DNA, 20 bp, synthetic construct
  source 1..20: mol_type = other DNA
ggctgtattcccctccatcg

SEQ ID NO: 6 - DNA, 22 bp, synthetic construct
  source 1..22: mol_type = other DNA
ccagttggtaacaatgccatgt

(19) United States
(12) Patent Application Publication
(10) Pub. No.: US 2024/0425824 A1
(43) Pub. Date: Dec. 26, 2024

(54) SCALABLE FABRICATION OF A SELF-ASSEMBLED CELL SHEET AND USES THEREOF

(71) Applicant: McMaster University, Hamilton (CA)

(72) Inventors: Ponnambalam Ravi Selvaganapathy, Dundas (CA); Maedeh Khodamoradi, Hamilton (CA)

(21) Appl. No.: 18/751,954

(22) Filed: Jun. 24, 2024

Related U.S. Application Data
(60) Provisional application No. 63/522,796, filed on Jun. 23, 2023.

(51) Int. Cl. C12N 5/071

(52) U.S. Cl. CPC C12N 5/0697; C12N 2513/00

(57) ABSTRACT

This disclosure relates to methods of fabricating self-assembled 3D cell sheets. In one embodiment, the cell sheets are formed by seeding cells to a monolayer density of at least 65% with a culture medium on to a substantially flat substrate having low surface energy and incubating cells on the substrate under time and temperature conditions to form a cell sheet.

### CROSS-REFERENCE TO RELATED APPLICATION

The present application claims the benefit of priority from co-pending U.S. provisional patent application No. 63/522,795 filed on Jun. 23, 2023, the contents of which are incorporated herein by reference in their entirety.

### REFERENCE TO SEQUENCE LISTING SUBMITTED ELECTRONICALLY VIA PATENT CENTER

The content of the electronically submitted sequence listing (Name: 3244-P72469US01_SequenceListing; Size 6200 bytes; Date of Creation: Jun. 24, 2024) submitted in this application is incorporated herein by reference in its entirety.

### FIELD

The present disclosure relates to tissue engineering and in particular, to the biofabrication of self-assembled cell sheets and uses thereof.

### BACKGROUND

Regenerative medicine is a field that aims to replace, repair, or regenerate damaged tissues or organs in the human body. Techniques such as cell-suspension injection, scaffold-based, and scaffold-free tissue engineering approaches have been developed to achieve this goal. Cell sheet tissue engineering is a scaffold-free technique that allows the fabrication of in vivo-like tissues, especially cell-dense and transparent tissues such as the heart and cornea. This technique addresses some limitations of other approaches, such as nonuniform cell distribution, inflammation, and autoimmunity at the implantation site. However, existing methods to fabricate cell sheets have limitations, such as a long culture period, extensive substrate treatment, fragility, and relatively small size of obtained cell sheets.

The background herein is included solely to explain the context of the disclosure. This is not to be taken as an admission that any of the material referred to was published, known, or part of the common general knowledge as of the priority date.

### SUMMARY

The present disclosure provides a simple and scalable fabrication method of a self-assembled cell sheet, which utilizes a low surface energy substrate such as untreated and non-cellular adherence polydimethylsiloxane (PDMS) mold to promote cell-cell connections instead of the cell-substrate attachment. Culturing cells in such a substrate can lead to the formation of a self-assembled and scaffold-free cell sheet within a short incubation time (FIG. 1A).

Accordingly, in an aspect of the disclosure a method of forming a cell sheet is provided, comprising:
seeding cells with a culture medium on to a substantially flat substrate, the substrate having low surface energy and/or ultralow adhesion; and
incubating cells on the substrate under time and temperature conditions to form a cell sheet;
wherein the cells are seeded on the substrate to a monolayer density of at least 65%.

In one embodiment, the cells are seeded on the substrate to a monolayer density of 90 to 100%.

In another embodiment, the incubation time ranges from 20 minutes to 10 hours.

In another embodiment, the incubation temperature ranges from 25° C. to 39° C.

In another embodiment, wherein the cell sheet is a 3-dimensional (3D) cell sheet.

In another embodiment, wherein seeding the cells comprises providing the cells to a mold, the bottom of the mold comprising the substrate.

In another embodiment, the mold defines the shape of the cell sheet.

In another embodiment, the substantially flat substrate comprises at least one topographical feature.

In another embodiment, wherein the substrate is selected from the group consisting of a silicone polymer, a fluoropolymer, a passivated or native polyolefin, a denser than water liquid, or a combination thereof.

In another embodiment, the silicone polymer comprises polydimethylsiloxane (PDMS), optionally pristine PDMS.

In another embodiment, the fluoropolymer comprises polytetrafluoroethylene (PTFE), the passivated or native polyolefin comprises polycarbonate, polystyrene, or polyethylene or the denser than water liquid comprises silicone oil, a pluronic solution, a sugar solution, an ionic liquid, or a deep eutectic solvent.

In another embodiment, the cells are selected from human cells or animal cells.

In another embodiment, two or more types of cells are seeded and incubated at different areas of the substrate.

In another embodiment, two or more types of cells are seeded and incubated at different areas divided by an insert on the substrate. Optionally, the insert is used to segment the substrate area for selective seeding of cells and is removed within 10 minutes to 1 hour of cell seeding.

In another embodiment, the insert has a thickness that ranges from 0.1 mm to 10 mm, optionally less than 4 mm.

In another embodiment, the culture medium is selected from the group consisting of a biological media, a synthetic media, or a combination thereof.

In another embodiment, the biological media comprises plasma, serum, or diluted tissue extract or the synthetic media comprises DMEM, EBM, EBM-2, F10, or F12.

Also provided is a method of constructing a multi-layer cell sheet, comprising: stacking two or more of the cell sheets produced by a method as described herein in a culture medium, wherein the cell sheets mutually fuse under time and temperature conditions.

In one embodiment, the time ranges from 10 minutes to 10 hours.

In another embodiment, the temperature ranges from 25° C. to 39° C.

In another embodiment, the culture medium is selected from the group consisting of a biological media, a synthetic media, or a combination thereof.

In another embodiment, the biological media comprises plasma, serum, or diluted tissue extract or the synthetic media comprises DMEM, EBM, EBM-2, F10, or F12.

In another embodiment, three cell sheets are stacked, the first cell sheet comprising cell types associated with the epidermis, the second cell sheet comprising cell types associated with the dermis and third cell sheet comprising cell types associated with the subcutaneous tissue. Optionally, the cell types associated with the epidermis comprise keratinocytes and mesenchymal stem cells, the cell types associated with the dermis comprise fibroblasts, vascular endothelial cells and mesenchymal stem cells and the cell types associated with the subcutaneous layer comprise adipocytes, muscle cells and endothelial cells.

Also provided is a method of constructing a two-dimensional (2D) or a three-dimensional (3D) cell construct comprising: overlapping the edges of two or more of the cell sheets produced by a method as described herein in a culture medium, wherein edges mutually fuse under time and temperature conditions.

In one embodiment, the time ranges from 10 minutes to 10 hours.

In one embodiment, the temperature ranges from 25° C. to 39° C.

In another embodiment, the culture medium is selected from the group consisting of a biological media, a synthetic media, or a combination thereof.

In another embodiment, the biological media comprises plasma, serum, or diluted tissue extract or the synthetic media comprises DMEM, EBM, EBM-2, F10, or F12.

Also provided is a use of a method as described herein for fabrication of a cell sheet for wound healing, regenerative medicine, drug discovery, or cultured meat.

In another aspect of the present disclosure, the method initially involves seeding cells with an appropriate culture medium on a substrate, wherein the seeded cells cover at least 70% of the substrate area. The seeded cells can be incubated with a desired incubation temperature and time in the culture medium to form a self-assembled cell sheet. Furthermore, two approaches are utilized to form a self-assembled cell sheet with two or more types of cells. One of the approaches is to use an external insert to separate the substrate into different areas and fill each area with a desired type of cells. After a period of incubation, the insert can be quickly removed, allowing cells to self-assemble and form a single multicellular cell sheet with a desired pattern (FIG. 1, Bc). Another approach is to design a confined substrate such as a dumbbell-shaped substrate, and each type of cells is seeded and incubated at a different area of the substrate to allow the formation of cell-cell connections. The formed sheet can be removed from substrate as a single multicellular cell sheet with a desired pattern (FIG. 1, Bd).

According to another aspect of the present disclosure, a thick structure can be constructed by stacking two or more self-assembled cell sheets layer by layer in a culture medium, wherein the layers can mutually fuse together under the appropriate time and temperature conditions (FIG. 1, Ba)

According to another aspect of the present disclosure, a two-dimensional (2D) or a three-dimensional (3D) structure can be constructed by overlapping the edges of two or more self-assembled cell sheets in a culture medium, wherein edges can mutually fuse together under the appropriate time and temperature conditions. (FIG. 1, Bb).

According to another aspect of the present disclosure, the fabrication method of self-assembled cell sheet and the method of constructing 2D structure, 3D structure and thick structure with multiple self-assembled cell sheets can be utilized to fabricate the tissue for regenerative medicine, drug discovery, or cultured meat.

Other features and advantages of the present disclosure will become apparent from the following detailed description. It should be understood, however, that the detailed description and the specific examples, while indicating embodiments of the disclosure, are given by way of illustration only and the scope of the claims should not be limited by these embodiments, but should be given the broadest interpretation consistent with the description as a whole.

### DRAWINGS

Certain embodiments of the disclosure will now be described in greater detail with reference to the attached drawings in which:

FIG. 1 shows the schematic illustration of cell sheet self-assembly formation (A), and other advanced capabilities of technique in terms of scalability, and formation of multicellular cell sheet constructs in exemplary embodiments of the disclosure.

FIG. 2 shows the cell density optimization in 1 cm diameter PDMS mold (A), formation of robust cell sheet after 5 h of incubation that could lead to easy manipulation and performing stacking procedure (B), monitoring the shrinkage of cell sheets over the longer culture period (C) in exemplary embodiments of the disclosure.

FIG. 3 shows the alternative cell lines that could self-assemble and form cell sheets in exemplary embodiments of the disclosure.

FIG. 4 shows the various morphologies of cell sheets in exemplary embodiments of the disclosure.

FIG. 5 shows the scalability of cell sheets to form larger constructs in exemplary embodiments of the disclosure.

FIG. 6 shows the positioning and assembly of cell sheets as building blocks of complex structures in exemplary embodiments of the disclosure.

FIG. 7 shows the stacking of cell sheets to form thicker constructs and monitoring their shrinkage behavior over 3 days of culture in exemplary embodiments of the disclosure.

FIG. 8 shows the patterning and co-culturing systems of cell sheets in exemplary embodiments of the disclosure.

FIG. 9 shows the HUVECs and fibroblast coculture system in exemplary embodiments of the disclosure.

FIG. 10 shows the live and dead micrographs of cell sheet constructs after 4 h and 1 day of culture in exemplary embodiments of the disclosure.

FIG. 11 shows microscopic evaluation of cell sheets to visualize the microstructure of cells by staining actin filaments of cytoskeleton and cells' nuclei in exemplary embodiments of the disclosure.

FIG. 12 shows histological assessment of cell sheets by staining cell sheets with H&E in exemplary embodiments of the disclosure.

FIG. 13 shows a chematic illustration of the scaffold-free cell sheet fabrication process. (A) (i) A sufficient number of cells are resuspended in their proper culture medium and poured into the untreated and non-adherent PDMS well. (ii) Microstructure evolution of cell sheet starting from seeding high cellular density in a PDMS mold, formation of cell-cell connections due to the expression of E-cadherin and finally ECM production and development of cell-ECM connections though integrin bindings. (B) Some of the capabilities of this technique are shown: (i) Cell sheets can be stacked on top of each other to form thicker tissue-like constructs. (ii) Cell sheets in various shapes can be made. (iii) They can be used as building blocks for complicated structures, like the word “MAC”.

FIG. 14 shows the effect of Effect of various ultralow adhesion substrates on self-assembly of fibroblast cells to form cell sheet. Both solid and liquid low surface energy and low adhesion surfaces cause self-assembly of cells to make continuous large area cell sheet constructs instead of cell aggregation while optimized cell density is seeded into the substrate.

FIG. 15 shows the macrostructure of cell sheets and their contraction behaviour. (A) Cells of various numbers were seeded into 1 cm diameter PDMS molds to optimize the seeding density for forming a robust and well-integrated flat sheet. A cell density equal to or above 0.8×10⁶ cells per mold supports sheet formation. However, considering the flatness and mechanical robustness of the sheets, the optimized density is determined to be 1.5×10⁶ cells per mold (D=1 cm). (B) The shrinkage behaviour of a single-layer cell sheet over 4 days reveals that the sheet contracts to approximately 60% of its initial dimension after 1 day. (C) The shrinkage patterns of single-layer and multi-layer constructs are compared. While all remain flat, increasing the number of sheets in the stacked constructs results in less shrinkage over the experimental period. (*, ***: P-value <0.05, and 0.001, respectively)

FIG. 16 shows the microstructure of cell sheets. (A) Confocal micrograph of self-assembled GFP-NIH/3T3 cells in the cell sheet format after 1 day of formation displays densely packed cells, emphasizing cell-cell connections. A live/dead image after 1 day reveals a minimal number of dead cells within the construct. (B) Evolution of the microstructure of cell sheets over time. (C) Phalloidin and DAPI stained constructs after 1 day of formation demonstrate the integrity of the cellular cytoskeleton in the sheets. Specifically, phalloidin stains the actin filaments of the cytoskeleton, while DAPI stains the nuclei. (D) H&E-stained cross-sections of single-layer cell sheets highlight the microstructure and time-related thickness changes in the samples. (E) Collagen staining of single-layer cell sheets was conducted using the PSR staining kit. The quantity of collagen expressed by cells in the cell sheet format over 5 days was quantified using ImageJ software. (**, ***: P-values <0.01 and <0.001, respectively).

FIG. 17 shows assays on single-layer and multi-layered cell sheets to investigate cellular behavior and functionality. (A) The metabolic activity of cells was assessed using ABA at 5 hours and one day post cell seeding. Values are normalized to the 2D group (measured 5 hours after seeding) which represents the monolayer culture containing the same number of cells as the cell sheets, but grown in regular multi-well cell culture plates. (B) The bar graph depicts the metabolic activity of single-layer, double-layer, and triple-layer cell sheet constructs. Their relative values are compared after one and two days of formation, with normalization to the single-layer cell sheet on day one. (C) The total protein content of single, double, and triple-layer constructs was determined using BCA and contrasted with the 2D monolayer culture of cells on plastic culture plates (D) Expression of cell-cell adhesion and cell-ECM adhesion markers in self-assembled cell sheet and regular 2D cell culture plates with same size as PDMS mold seeded with same number of cells after 1 day. (**, ***: P-value <0.01, and 0.001, respectively)

FIG. 18 shows various capabilities of the developed biofabrication technique. (A) Generation of cell sheets from alternative cell lines. (B) Production of large-area cell sheets ranging in diameter from 0.5 cm to 4.8 cm. (C) Creation of cell sheets with diverse morphologies. (D) Formation of complex structures by assembling individual building units into a more intricate unit.

FIG. 19 shows patterning and coculturing strategies of cells in cell sheet format. (A) Fluorescent micrographs illustrate five different patterning techniques: (i) A bi-layer system, developed via sequential deposition of cell layers in the same PDMS well with 30-minute intervals, displays distinct layers of red and green labeled cells. These layers, while distinguishable, combine to form a single unit. (ii) The mixed co-culture system showcases the potential for achieving a homogeneous distribution of heterogeneous cells within a cell sheet construct. (iii) The use of a confined PDMS well, combined with sequential cell deposition into each well section, permits a co-culture patterning strategy. (iv) External inserts, which partition the PDMS well seeding area into two separate compartments, facilitate a half/half co-culturing approach. Cells are seeded into each section simultaneously, incubated for 15 minutes, and then the insert is removed. This allows cells from both sides to connect and create a seamless interface. (v) The marbling patterning technique employs: v1) free hand drawing by sequential deposition of different color tagged cells using pipettes to draw the desired pattern, and v2) using external insert with a marbling structure and deposition. (B) One application of coculturing is the creation of perivascular tissue constructs. A coculture sheet consisting of GFP-3T3 and RFP-HUVECs was fashioned using the mixed strategy, developed a perivascular fibroblast construct after 4 days of incubation. (C) Bilayer coculture system also used to make a prevascularized fibroblast sheet by sequentially deposition of fibroblasts (first layer), and then HUVECs on top of it (second layer).

FIG. 20 shows a schematic illustration of patterning and co-culturing strategies developed using the cell sheet biofabrication technique described herein. (a) External inserts with a separation wall of 600 μm were employed to facilitate the formation of a half/half co-culturing system. (b) A confined PDMS mold in a dumbbell shape enables the positioning of differently labeled cells within a single mold. (c) Sequential deposition of cells on top of each other results in the formation of bilayer co-cultured constructs, where two distinct layers are evident, yet seamlessly fused together.

### DETAILED DESCRIPTION

### I. Definitions

Unless otherwise indicated, the definitions and embodiments described in this and other sections are intended to be applicable to all embodiments and aspects of the present disclosure herein described for which they are suitable as would be understood by a person skilled in the art. It is also to be understood that the terminology used herein is for the purpose of describing particular aspects only and is not intended to be limiting.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies. In addition, all ranges given herein include the end of the ranges and also any intermediate range points, whether explicitly stated or not.

As used in this disclosure, the singular forms “a”, “an” and “the” include plural references unless the content clearly dictates otherwise.

In embodiments comprising an “additional” or “second” component, the second component as used herein is chemically different from the other components or first component. A “third” component is different from the other, first, and second components, and further enumerated or “additional” components are similarly different.

The term “and/or” as used herein means that the listed items are present, or used, individually or in combination. In effect, this term means that “at least one of” or “one or more” of the listed items is used or present.

The abbreviation, “e.g.” is derived from the Latin exempli gratia and is used herein to indicate a non-limiting example. Thus, the abbreviation “e.g.” is synonymous with the term “for example.” The word “or” is intended to include “and” unless the context clearly indicates otherwise.

The term “construct” or “cell construct” as used herein refers to both a single cell sheet and a plurality of associated cell sheets, for example a multilayer cell sheet.

It will be understood that any component defined herein as being included may be explicitly excluded by way of proviso or negative limitation, such as any specific compounds or method steps, whether implicitly or explicitly defined herein.

### II. Compositions and Methods of the Disclosure

The present disclosure shows that large-area cell sheets (for example, >5 cm in diameter) that may be 5-10 cells thick can be produced by exploiting cells' preferential adhesion to each other over the substrate, without requiring specialized thermoresponsive substrates or harsh delamination processes. Unlike prior art methods, a 3-dimensional (3D) cell sheet is formed. Further, these self-assembled sheets are mechanically robust, compatible with various cell types, and can secrete their own ECM. In addition, by stacking these sheets, thicker constructs can be created or a pick and place technique can be used for intricate patterning. In an in vivo porcine thermal injury model, UC-MSC cell sheets demonstrated biocompatibility and reintegrated well into the wound bed. This approach closely mimics native tissue microstructures and is applicable to regenerative medicine, meat cultivation, and in vitro tissue modeling.

Accordingly, the present disclosure provides a method of forming a cell sheet, comprising:
seeding cells with a culture medium on to a substantially flat substrate, the substrate having low surface energy and/or ultralow adhesion; and
incubating cells on the substrate under time and temperature conditions to form a cell sheet;
wherein the cells are seeded on the substrate to a monolayer density of at least 65%.

As used herein, the term “substantially flat substrate” refers to a planar or largely planar substrate such as the bottom surface of a cell culture plate or well. Different substrate shapes are contemplated. In some embodiments, the substrate is circular (for example, the bottom surface of a cell culture plate or well. In other embodiments, the substrate is oval, rectangular, square, dumbbell or irregularly shaped.

In some embodiments, the substrate is the bottom surface of a mold. As used herein, the term “mold” refers to a vessel designed to constrain the shape of the cell sheet. The mold can be a cell culture plate or well, or it can be of any shape of interest. In one embodiment, the mold defines the shape of the cell sheet.

In some embodiments, the substrate has a low surface energy and/or ultralow adhesion. As used herein, the term “low surface energy” refers to a surface energy of less than low surface energy 36 mJ/m².

In some embodiments, the substrate does not have any anchoring locations for the cells to attach to.

In some embodiments, the substantially flat substrate comprises at least one topographical feature. The topographical features are selected based on the desired shape of the cell sheet. Topographical features include, but are not limited to, grooves (for example, straight lines or chevron shaped), bumps (for example, arrays of bumps) or pillar-type indentations.

In some embodiments, the substantially flat substrate is a semi-solid or liquid substrate.

In some embodiments, the substrate comprises or is a silicone polymer (for example, polydimethylsiloxane (PDMS)), optionally pristine PDMS or untreated PDMS). As used herein, the term “pristine PDMS” refers to PDMS with low surface energy, for example 19-21 mJ/m².

In some embodiments, the substrate comprises or is a fluoropolymer (for example, polytetrafluoroethylene (PTFE)), a passivated or native polyolefin (for example, polycarbonate, a polystyrene, or a polyethylene), a denser than water liquid (for example, silicone oil, a pluronic solution, a sugar solution, an ionic liquid, or a deep eutectic solvent), or a combination thereof.

The cells may be seeded on the surface by any method known in the art. In some embodiments, seeding comprises replating from a cell culture plate.

The cells are optionally seeded such that they cover at least 65%, 70%, 75%, 80%, 85% or 90% of the substrate area. or such that they cover about 65%, 70%, 75%, 80%, 85% or 90% of the substrate area. As used herein, percentage cover or coverage percentage of the surface area of a substrate that is covered with cells.

In some embodiments, the cells are seeded to at least or about 65%, 70%, 75%, 80%, 95%, 90%, 95%, 99% or 100% monolayer density. In some embodiments, the cells are seeded to 90% to 100% monolayer density. As used herein, the term “monolayer” refers to refers to a layer of cells in which no cell is top of another, but are all side by side and the term “monolayer density” refers to the density of cells when they are in a monolayer.

In some embodiments, the cells are seeded to a density of 1.5 to 2.0×10⁶ cells/cm² of the surface area of the substrate.

The cells are incubated on the substrate under time and temperature conditions to self-assemble into a cell sheet. In some embodiments, the time ranges from 20 minutes to 10 hours, optionally 3 to 5 hours.

In some embodiments, the incubation temperature ranges from 25° C. to 39° C., optionally 37° C. or about 37° C.

In further embodiments, the cells are incubated in 2.5% to 10% CO₂, optionally 5% CO₂ or about 5% CO₂.

One advantage of the methods described herein over prior art methods is that that a 3-dimensional (3D) cell sheet is formed. Accordingly, in one embodiment, the cell sheet is a 3D cell sheet. In another embodiment, the cell sheet is at least 4, 5, 6, 7, 8, 9, 10, 11, 12, 13, 14 or 15 cells thick, optionally 5-10 cells thick.

In some embodiments, the cell sheet is greater than, 3, 4, 5, 6, 7, 8, 9, or 10 cm in diameter.

The cells may be cultured on any typical cell culture surface and in appropriate culture medium prior to the plurality of cells being seeded on the substrate. For example, cells may be grown on porous particles in a bioreactor or in a bioreactor without a scaffold. In some embodiments, the cells are grown to 80-90% confluence prior to seeding.

After culturing, the plurality of cells are optionally dissociated before being seeded on the substrate. In some embodiments, for example when the cells grown without a scaffold, the cells do not need to be dissociated prior to plating. Cells may be dissociated by any method known in the art, including, but not limited to dissociation using trypsin or other enzymes such as dispase or collagenase, non-enzymatic dissociation agents Accutase™, or by application of mechanical forces such as scraping.

The cells are seeded on the substrate along with an appropriate culture medium. As used herein, the term “culture medium” refers to a liquid or semi-solid that support the growth of cells. A culture medium that is suitable for the specific cell type(s) of the cells may be used.

In one embodiment, the culture medium is a liquid medium.

In some embodiment, the culture medium is a biological medium, a synthetic medium, or a combination thereof.

Examples of biological mediums include, but are not limited to mediums comprising plasma, serum, lymph, amniotic fluid, pleural fluid, growth factors, hormones, crude protein fractions, recombinant proteins, protein hydrolysates, tissue extracts or combinations thereof.

In another embodiment, the culture medium comprises a basal medium and supplements selected from the group consisting of plasma, serum, lymph, amniotic fluid, pleural fluid, growth factors, hormones, crude protein fractions, recombinant proteins, protein hydrolysates, tissue extracts or a combination thereof. Examples of synthetic medium useful in the present methods include, but are not limited to, Dulbecco's Modified Eagle Medium (DMEM), supplemented for example with 10% V/V fetal bovine serum (FBS) and 1% Penicillin-Streptomycin, Endothelial Basal Medium (EBM), EBM-2 medium, Ham's F-10 Nutrient Mixture (F-10), Ham's F-12 Nutrient Mixture (F12) and McCoy's medium supplemented for example with 15% V/V fetal bovine serum (FBS) and 1% Penicillin-Streptomycin.

In some embodiments, the cells comprise animal or human cells. In some embodiments, the cells comprise hepatocytes, pancreatic Islet cells, fibroblasts, chondrocytes, bone cells, osteoblasts, ondontoblasts, skin cells, keratinocytes, melanocytes endothelial cells, exocrine cells, smooth or skeletal muscle cells, myocytes, preadipocytes, adipocytes, ectodermal cells, ductile cells, kidney cells, intestinal cells, parathyroid and thyroid cells, nerve cells, ocular cells, retina cells, integumentary cells, immune cells, vascular cells, heart cells (for example cardiomyocytes), bone marrow cells, neural cells (for example gila and astrocytes), placental cells, pluripotent cells and stem cells, cancer cells and tumor cells, or combinations thereof.

In some embodiments, the cells comprise myoblasts, muscle cells, endothelial cells, fibroblasts, neuroblastoma cells, preadipocytes, adipocytes, or a combination thereof. In some embodiments, the plurality of cells comprises myoblasts, preadipocytes, or a combination thereof. In some embodiments, primary animal cells including satellite cells (muscle stem cells), mesenchymal, embryonic, or induced pluripotent stem cells can be considered for the muscle progenitor cells, while adipose tissue derived stem cells or mesenchymal stem cells can be considered for the fat progenitor cells. Cell lines for both these cell types in meat as well as other cell types, including endothelial cells comprising blood vessels and cell types forming the connective tissue, can be created through genetic or chemical induction and may a better option to reduce variability and contamination risks associated with primary cell harvest. In some embodiments, the plurality of cells further comprises plant cells.

In some embodiments, the cells comprise keratinocytes, mesenchymal stem cells, fibroblasts, vascular endothelial cells, mesenchymal stem cells, adipocytes, muscle cells or endothelial cells.

In some embodiments, the cells comprise different cell types existing as a homogenous mixture or substantially homogenous mixture, resulting in a cell sheet comprising a homogenous mixture or substantially homogenous mixture. In some embodiments, the plurality of cells comprises different cell types which are deposited such that the different cell types spatially separated within the cell sheet. In some embodiments, different cell types are deposited in a defined pattern to obtain spatial separation within the construct.

In some embodiments, the cell sheet retains a defined shape by controlling the location of the initial cell seeding. In some embodiments, controlling the location of the initial cell plating comprises blocking cell growth in a defined area of the substantially flat substrate.

In one embodiment, the cells are plated in a pattern that defines at least one void. A plurality of cell sheets may be aligned and stacked to provide a three-dimensional void in the construct. Optionally, the three-dimensional void comprises or is a channel, an inlet or an outlet.

In another embodiment, multiple cell types can be used and plated in pattern. For example, in some embodiments, cells can be plated in a pattern on top of a preformed, base cell sheet. The cells are of the base cell sheet are optionally of the same cell type or a different cell type that the cells plated on top.

In some embodiment, a cell sheet comprising a heterogenous mixture of cells is formed through the use of at least one insert, or barrier, which separates the substrate into more than one distinct sections. Different cell types may then be seeded in the different sections. In some embodiments, the insert comprises polylactic acid (PLA) and optionally has a thickness of 0.1 mm to 10 mm. optionally 500-700 μM, about 600 μM. In some embodiments, the insert has a thickness of less than 400 μM, 300 μM, 200 μM or 100 μM. The insert may be removed after seeding, for example after the cells have settled on the substrate. In some embodiments, the insert is removed within 10 minutes and one hour or after at least 5, 10, 15 or 20 minutes.

In some embodiments, the method of further comprises stacking two or more detached cell sheets to form a multilayer construct, optionally a double, triple or quadruple layer construct. The multilayer construct is optionally formed by picking individual cell sheets up (for example via tweezers or a robot handler) and placing one on top of the other. In some embodiments, cell culture medium is aspirated from the sheets after stacking. Following medium removal, the cell sheets are optionally allowed to settle for at least 5, 10. 15, 20 or 25 minutes, then cell culture medium may be added back. Thus, in some embodiments, a pick and place operation is used to overlap individual cell sheets precisely to get complex 2D and 3D arrangements. The cell sheets can then fuse at the contact points forming a monolithic structure.

In some embodiments, the cells in the sheet form cohesive linkages with each other after stacking. In some embodiments, the layers of sheets form a mechanically robust construct. Each of the cell sheets can have a different combination of cell types and shape. Stacking two or more detached cell sheets to form a multilayer construct method may be used to create complex 3D cell culture systems that can mimic natural tissue compositions.

The method described herein may be used to make meat-like tissues of various sizes and thicknesses. Accordingly, in one embodiment, the plurality of cells comprises adipocytes, skeletal muscle cells and/or other connective tissue cells normally present in muscle tissue. Mixtures of different cell types allow fine tuning of fat and protein content in a fast and scalable fashion. For example, constructs made from only myoblasts may be equivalent to lean meat with comparable protein and fat content, while incorporating adipocyte cells in different ratios to myoblasts and/or treatment with different growth media can result in a 5% (low fat meat) to 35% (high fat meat) increase in the fat content.

The method described herein may also be used to create tissue for use in wound healing. Accordingly, in one embodiment, a construct for use in wound healing is provided comprising a three-layer cell sheet construct, with each cell sheet/layer comprising cell types associated with the epidermis, dermis and the subcutaneous tissue. Each layer may be constructed separately as a cell sheet and then assembled on top of each other where they will fuse. The assembled sheet can then be placed on a cleaned wound site where upon it will fuse with the preexisting tissue underneath.

In one embodiment, the epidermis layer is composed of a combination of keratinocytes and mesenchymal stem cells that are differentiated to form the epidermis layer, the dermis layer is composed of combination of fibroblasts, vascular endothelial cells and mesenchymal stem cells and the subcutaneous layer is composed of adipocytes, muscle cells and endothelial cells.

Also provided herein is a cell construct comprising a plurality of differentiated or partially differentiated cells formed according to the method described herein. In some embodiments, the construct is used in vivo or in vitro for research and development. In some embodiments, the construct is used in vivo for cell therapy. In some embodiments, the construct is used to prepare artificial meat products (also known as cultivated meat).

In some embodiments, the construct is used in vitro for research and development, such as for modeling cellular interactions in understanding disease and drug discovery. In some embodiments, the construct is used in vivo for cell therapy, such as tissue grafts and artificial organs for implantation.

The constructs described herein can be used for drug screening. Accordingly, also provided herein is a method for screening for activity of a compound of interest comprising treating a construct as described herein with a compound of interest and observing the effect of the compound on the plurality of cells. For example, a compound of interest may be screened for its effect on the growth rate of the cells, the viability of the cells and/or protein expression in the cells. In one embodiment, different doses of the compound of interest may be studied. The compound of interest is optionally a drug candidate, including for example, a small molecule or a biologics.

The constructs described herein can also be used as in vivo or in vitro bioreactors where cells producing specific biomaterials for example, a protein (for example, an antibody), peptide, hormone (for example, insulin), nucleic acid or lipid are included in the biocompatible gel. Accordingly, in such an embodiment, the methods described herein further comprise culturing the construct and isolating a biomaterial of interest.

The constructs described herein can be further used in regenerative medicine and/or wound healing. Accordingly, in such embodiments, the methods described herein further comprise administering the construct to a subject in need thereof. The wound is optionally a cut, burn or area which has been surgically excised.

In some embodiment, a construct for use in wound healing comprises a three-layer cell sheet construct, with each cell sheet/layer comprising cell types associated with the epidermis, dermis and the subcutaneous tissue. Each layer may be constructed separately as a cell sheet and then assembled on top of each other where they will fuse. The assembled sheet can then be placed on a cleaned wound site where upon it will fuse with the preexisting tissue underneath.

Also provided herein are methods of using the constructs of the disclosure as in vitro experimental models.

### EXAMPLES

The following non-limiting examples are illustrative of the present disclosure:

### Example 1

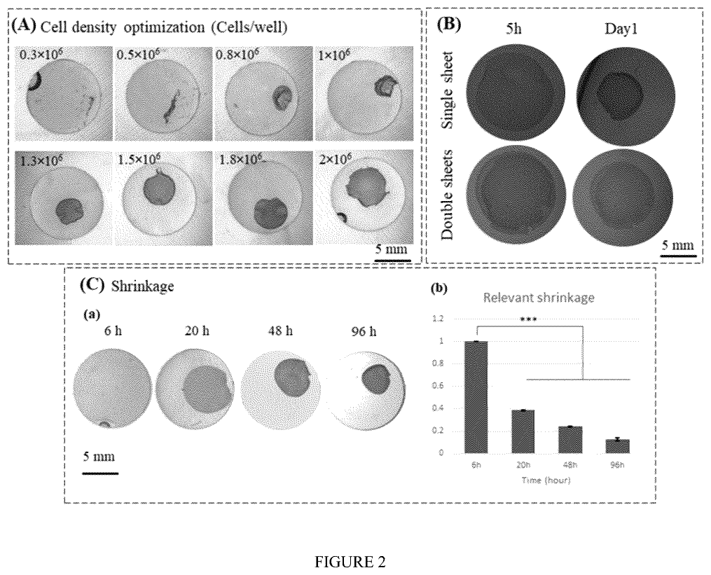

Cell sheet formation process: 3T3 fibroblasts were cultured in their growth media, DMEM (high glucose, L-glutamine, sodium pyruvate), supplemented with 10% V/V Fetal bovine serum (FBS, gibco), and 1% V/V penicillin/streptomycin. Meanwhile, a PDMS mold was created. Briefly, a negative of the desired shape was 3D printed to make a proper ABS master mold for PDMS casting. In this study, various shapes of mold were designed, such as circular, cross, dumbbell-shape, star, and square, to show the feasibility of this technique in creating different morphologies of cell sheets. Then, SYLGARD 184 elastomer and its curing agent were mixed in a ratio of 10:1 and degassed for 1 h minutes in a vacuum desiccator. Next, the PDMS solution was poured into the ABS master mold and kept at 70° C. for 2 h to complete the PDMS solidification. The PDMS mold was then removed from its master mold and sterilized using 70% ethanol to be prepared for cell culture. While 3T3 fibroblasts reached about 80% of confluency, they were detached from the tissue culture plate and counted. To optimize the cell density per each PDMS mold that would lead to the formation of the robust and thin cell sheet, different cell densities ranging from 0.3×10⁶ to 2×10⁶ cells per well were taken and poured into the circular PDMS mold with a 1 cm diameter. As FIG. 2A shows, the cell densities below 1.3×10⁶ cells/well did not support the formation of a flat and robust cell sheet, while by increasing the cell density to 1.3×10⁶ cells/well and higher, a continuous and planar self-assembled construct was formed after 1 day of incubation at 37° C. and 5% CO₂. This outcome also supports the mathematical calculation based on the division of the surface area of PDMS mold by approximated circumferential area of trypsinized cells, which indicates that to have a confluent monolayer of cells that cover the whole surface of PDMS substrate, 1.3-1.5×10⁶ cells/well is required. The optimal cell density was set to 1.5×10⁶ cells/well, and it is shown that the proper timing for 3t3 fibroblast cells to make a robust self-assembled cell sheet is as little as 4-6 h. FIG. 2B shows the formation of cell sheet after 5 h of culturing 3t3 fibroblasts in a PDMS mold and shows its enough mechanical robustness to be easily manipulated by removing it from the PDMS mold using a tweezer and replacing it in a regular tissue culture well plate to gain enough nutrition and also staking them to make a thicker construct. Besides, as it is indicated in FIG. 2C, cell sheet constructs were undergone uniform shrinkage by keeping them in culture for a longer period, and their area reduced by 60% after 1 day of culture, while they remained flat and circular.

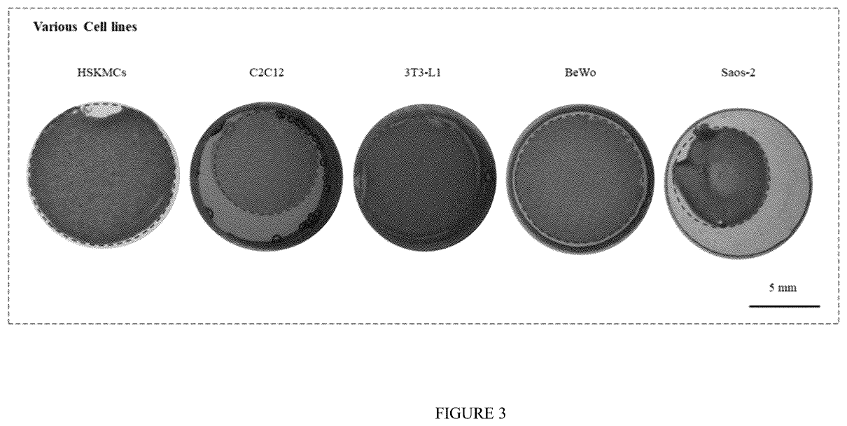

Alternative fusing cell lines that formed self-assembled cell sheets: Six different cell types have been used to form cell sheets in this study. In addition to 3T3 mouse fibroblasts, C2C12 mouse myoblasts, 3T3-L1 mouse preadipocytes, HSKMC human primary skeletal muscle cells (HSKMCs), SaOs-2 human osteosarcoma cell line, and BeWo human placenta choriocarcinoma cells were grown in their specific culture medium. Mouse myoblast C2C12 cells were grown in Dulbecco's Modified Eagle Medium (DMEM) (with L-glutamine and high glucose, Gibco), supplemented with 10% v/v heat-inactivated fetal bovine serum (HI-FBS, Canadian origin, ThermoFisher), and 1% v/v penicillin-streptomycin (10,000 U/ml, ThermoFisher). Mouse preadipocytes 3T3-L1 cells were cultured in DMEM+GlutaMAX (low glucose and with sodium pyruvate, Gibco) with 10% V/V HI-FBS and 1% V/V penicillin-streptomycin. Primary human skeletal muscle cells were grown in their ready-to-use growth medium (PromoCell, Catalogue number C-23060) and differentiated using the ready-to-use differentiation medium (PromoCell, Catalogue number: C-23061). SaOs-2 cells were cultured in McKoy's medium supplemented with 15% FBS and 1% pen/Strep, and BeWo cells were grown in Ham's F-12K medium containing 10% FBS and 1% pen-strep. The self-assembly ability of cells to form a stable cell sheet is dependent on the cell type (FIG. 3). Human skeletal muscle cells (HSKMC), mouse myoblasts (C2C12), mouse preadipocytes (3T3-L1), Human placenta choriocarcinoma (BeWo), and Human Osteosarcoma (Saos-2) formed robust cell sheets by interconnecting with adherent junctions, while other cell types such as human umbilical vein endothelial cells and human cornea epithelial cells did not form robust self-assembled cell sheets. As can be seen in FIG. 3, the construct shrinkage is also different among various cell lines, while they all self-assembled and formed cell sheet constructs.

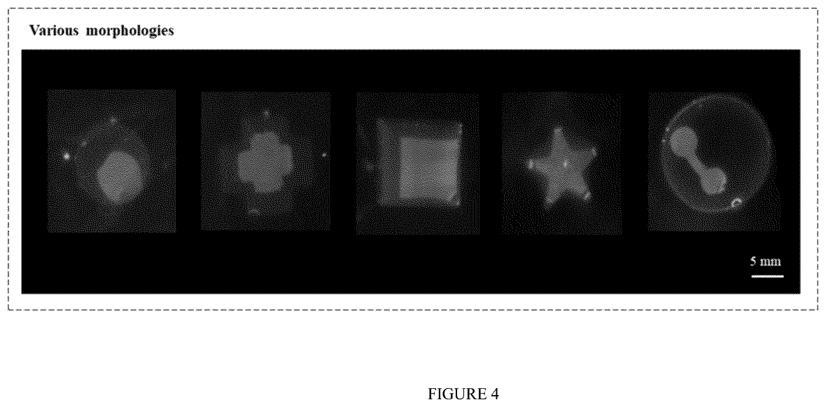

Formation of various cell sheet morphology: As FIG. 4 shows, making cell sheets in various shapes is possible by adopting our developed technique. In this regard, PDMS molds were made with different shapes to investigate the feasibility of this cell sheet engineering technique to determine the final shape of the cell sheets. The cell sheet constructs are consolidated uniformly in all directions. Interestingly, they retained their shape after removing from their confined space and keeping in a 3 cm tissue culture plate for three days. The capability of forming cell sheets in different sizes and shapes has many applications in biomedical engineering. In other words, this technique enables the production of custom-made cell sheets and 3D structures in a rapid, non-expensive, and simple way. The sites of injury or required tissue regeneration patterns could be scanned to design a master mold. Then proper PDMS mold could be made based on the desired pattern, and patient-specific cells can be used to produce the self-assembled and free-standing cell sheets implanted into the target tissue to support its regeneration.

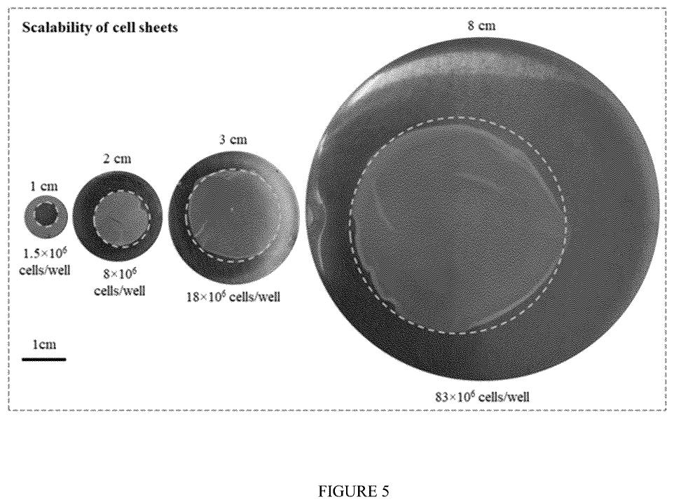

Making larger sheets: To show the feasibility of this technique to be scaled up and form larger and more complex cell sheet constructs, three different approaches have been employed. First, circular PDMS molds with various diameters ranging from 1 to 8 cm were fabricated. Then, the optimal cell density per each well was calculated and cultured in its respective size of PDMS mold. The cells were kept incubated for 20 h, and as FIG. 5 shows, robust cell sheet constructs with different sizes were fabricated. This capability of our developed technique is of great interest, especially in the case of skin tissue engineering and skin grafts due to the large wounded and burnt areas that require fast closure and promoting of the healing procedure.

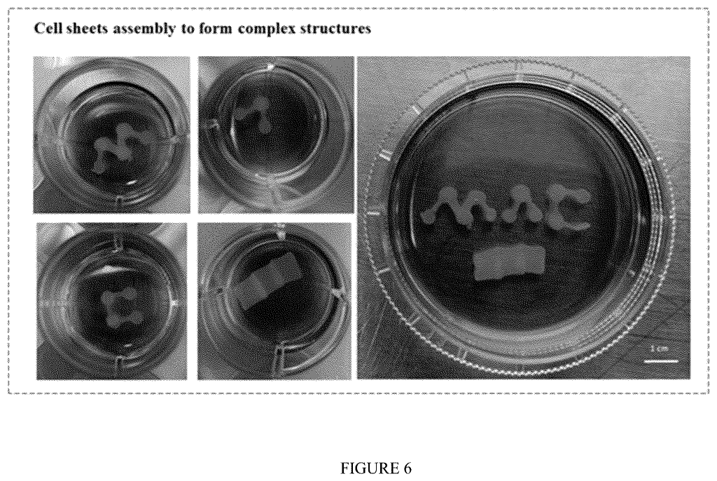

Assembly of sheets to make complicated constructs: The second approach to scale up the cell sheet constructs is positioning and assembly of cell sheets as the building blocks to make larger and more complex structures. To this purpose, multiple dumbbell-shaped and square cell sheet constructs were fabricated and precisely positioned in a way to make multiple and complex constructs after cell sheet fusion was completed at the sites of overlapping (FIG. 6). In more detail, after removing the sheets from their respective PDMS mold, they were kept in a regular 12-well plate, and all the assembly procedure was performed in a high volume of growth medium. To ensure that cell sheets are attached to each other, the growth medium was discarded gently to avoid the movement of cell sheets from their associated position. Then, they were kept incubated at 37° C. for 30 minutes, and then the proper volume of growth medium was gently added to the well plates. This complex structure can be easily removed from its container using a tweezer and placed in another tissue culture plate. They are interestingly robust enough to handle and retain their shape during the culture period.

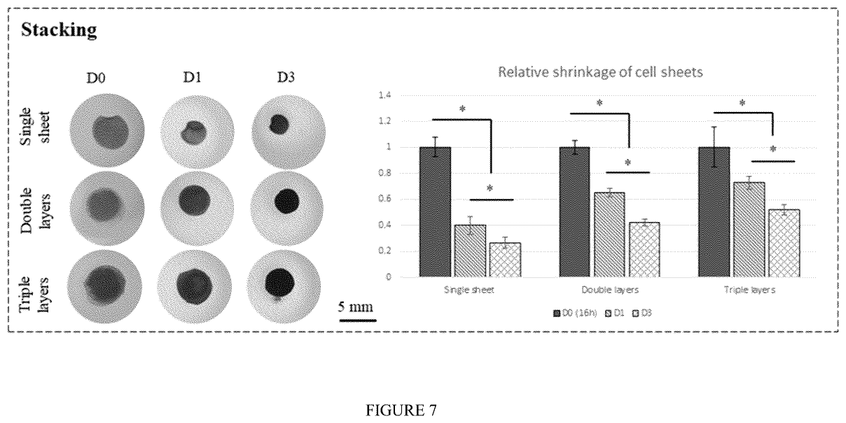

Stacking cell sheets: In order to make thicker tissue-like constructs, stacking cell sheets on top of each other was performed as a third option to scale up the constructs. As shown in FIG. 7, single, double, and triple layers constructs were formed, and their shrinkage over time was monitored and measured. Briefly, cell sheets were made by seeding cells in the PDMS mold. After the self-assembly process was completed, the cell sheets were moved to the regular 12-well plate. Then, the stacking procedure was performed in plenty of growth medium. While cell sheets were stacked and aligned on top of each other, the excess amount of growth medium was taken out slowly, and the constructs were kept incubated at 37° C. for 30 minutes to let the sheets begin the fusion. Then, the proper volume of growth medium was added to the constructs and kept incubated. As the bar graph shows, by increasing the number of stacked sheets in the construct, its shrinkage over time would be reduced. This reduction can be occurred due to the formation of new cell-cell, cell-ECM, and ECM-ECM adhesion between the top cellular layer and the bottom cell sheet. In other words, these layers act as the suitable substrate for the cellular component of other layers to be attached and make interconnections that would lead to compromising of cellular contraction forces toward the center of the sheet.

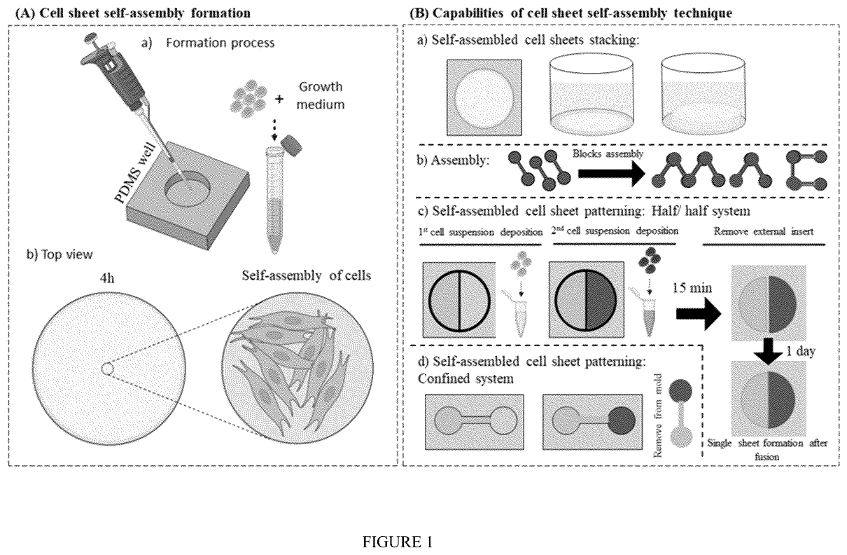

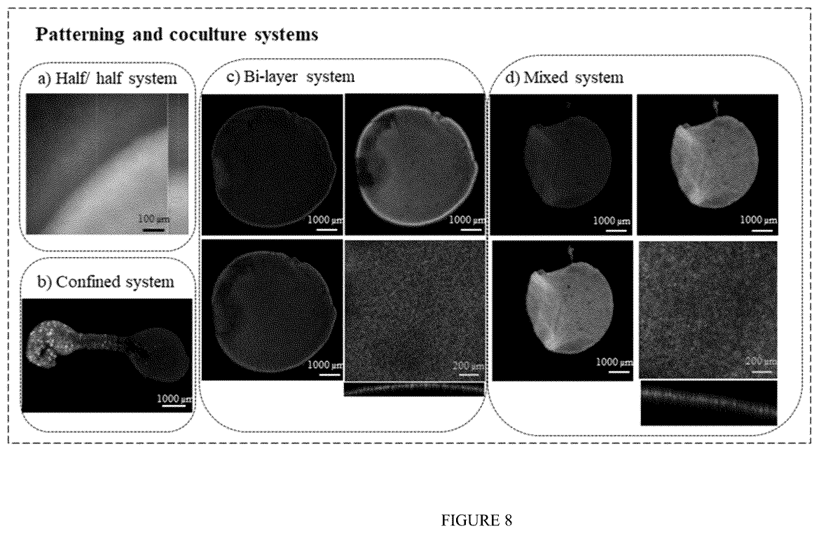

Patterning and making multicellular cell sheets: Cell patterning and employing co-culture strategies are challenging approaches using cell sheet engineering methods, while those are required to make complex tissue-like structures. Four strategies were used in this study to investigate whether our biofabrication technique would support cellular patterning and co-culturing different cell types in a cell sheet format. First, a 3D-printed PLA external insert with a separation wall thickness of 600 μm was made to divide the PDMS chamber area into two distinct sections (FIG. 1-Bc). Then, each section was filled out by different cell suspensions. After 15 minutes of incubation, the insert was removed slowly while the cells settled down, so cell-cell connections at the fine boundary between these sections were made to connect them and form a contiguous cell sheet. FIG. 8a shows the perfect binding of DiO and Dil-stained 3T3 cells to make a boundary where two sheets reach each other. The second technique is subsequently adding different cellular suspensions to a confined PDMS mold. The dumbbell-shaped PDMS structure was chosen as the constrained shape of the mold, and DiO/DiL-stained 3t3 fibroblast cells were poured into each section at intervals of 15 minutes. A well-defined border between these two sections can be seen in FIG. 8B, which shows a single continuous multicellular cell sheet construct.

Another co-culture system was also developed to make bilayer cell sheet constructs. To this purpose, first, DiL-stained 3t3 cells were added to the PDMS mold and followed by 30 minutes of incubation at 37° C. Then, the excess volume of the culture medium was slowly taken out, and DiO-stained cell suspension was gently added to the PDMS mold on top of the previous layer of cells. FIG. 8C shows the formation of two continuous and well-distinguished cell sheets on top of each other after 20 h of incubation. The last co-culture system was introduced by making a homogenous mixture of DiO and DiL stained 3t3 fibroblasts with a 1 to 1 ratio and co-culturing them in the PDMS mold. FIG. 8D represents the microscopic structure of these mixed co-culture cell sheets after 20 h of incubation at 37° C. As is clear in the cross-section view of micrographs, a well-distinguished interface between the top and bottom layer of construct in the Bilayer co-culture system could be seen, while a homogenous mixture of cells in the mixed system is detected by looking at the cross-section view. Altogether, the outcomes of these experiments showed the possibility of cell patterning and co-culture of different cell types in a self-assembled cell sheet format.

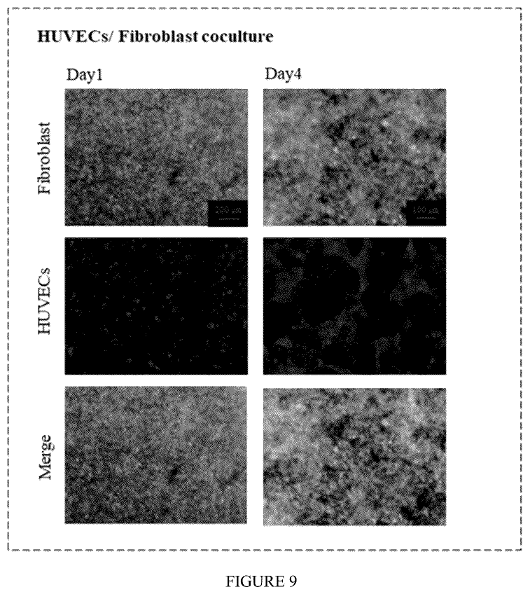

Perivascular cell sheet formation: Human umbilical vein endothelial cells (HUVECs) did not form continuous cell sheet constructs after culturing them in the PDMS mold. The HUVECs constructs were disaggregated while removing them from the PDMS mold, and they were not robust enough to support the contiguous cell sheet handling. But it is still possible to co-culture HUVECs with other cell types, such as fibroblast cells, and make perivascular tissue-like constructs (FIG. 9). To make this perivascular cell sheet constructs, a combination of red fluorescent protein-tagged (RFP)-HUVECs, and green fluorescent protein-tagged (GFP)-3t3 fibroblast cells with the ratio of 1:4, respectively, were suspended in the fibroblast growth medium and then added to the 1 cm circular PDMS mold. This co-culture system was kept incubated at 37° C. and 5% CO2 for 4 days, and its microstructure evolution was monitored using Zeiss fluorescent microscopy. FIG. 9 represents the formation of a single and continuous construct out of fibroblast and HUVECs. It is also interesting to see the self-assembly of HUVECs in the construct after 4 days of culture that prove the possibility of formation of microvasculature inside the perivascular construct. Self-assembly of HUVECs to form microvasculature would possibly result in increasing the efficiency of graft survival after implantation at the site of injury by making an interconnecting network with the body vascular bed. Moreover, it could reduce the chance of necrotic core formation within the thicker constructs by incorporating these perivasculature constructs into the stacked cell sheet constructs.

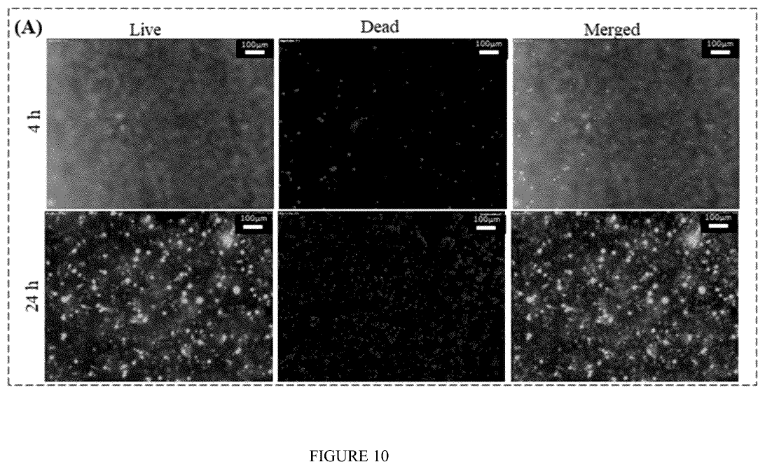

Cellular viability assessment in cell sheets: To investigate the cellular viability inside the self-assembled cell sheet constructs, live and dead assessments were performed using Calcien-AM and Ethidium homodimer to visualize live and dead cells, respectively, after 4 hours and one day of culturing 3t3 fibroblasts inside the PDMS mold. Calcien-AM and Ethidium homodimer were diluted in 3T3 growth media with ratios of 1:2000 and 1:500, respectively, added to the cell sheets, and incubated for 30 minutes. Followed by washing the dyes, the live and dead imaging was captured using an inverted fluorescent microscope (Olympus) and using proper filters of TEXRED and FITC. The fluorescent images in FIG. 10 show the presence of a very low number of dead cells in the construct after 4 h, while the number of dead cells increased after one day, which could be the result of insufficient transport of nutrients and oxygen due to the shrinkage and transformation of sheets to the thicker 3D constructs. The results indicate that while there were no suitable cell-surface attachment sites on the culture surface, cells stay alive by attaching to each other and making strong cell-cell connections.

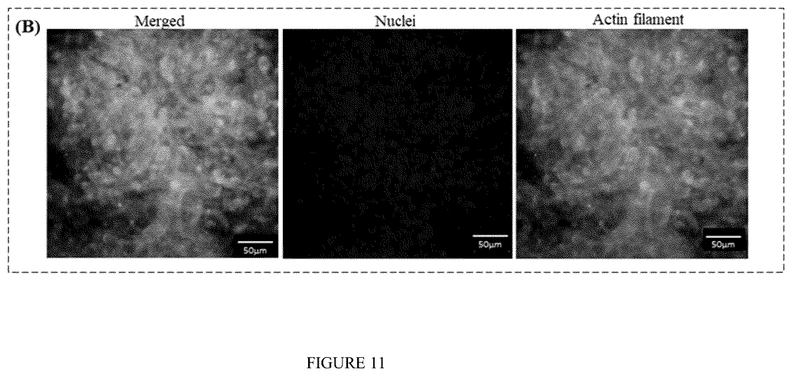

Morphological assessment of cell sheets: Morphological analysis was performed to visualize the microstructure of the cells in the cell sheet constructs. Alexa Fluor™ 488 Phalloidin stock solution (300 units dissolved in 1.5 ml methanol) and DAPI stock solution (10 mg/mL in DI water) were diluted in 0.1% Triton X-100 in DPBS with the ratios of 1:1000 and 1:2000, respectively. Cell sheets were fixed after 30 minutes of immersion in 4% PFA solution and washed three times with PBS. Then, cell sheets were stained with 500 μL of diluted Phalloidin and DAPI in RT for 1 hr. The microscopy was conducted by using Ti2 confocal microscopy with proper filters. As FIG. 11 shows, a compact aggregate of nuclei and integrated actin filament structures indicates a perfect formation of a cell-rich construct and well-formed cell-cell connection without using any external scaffolding material.

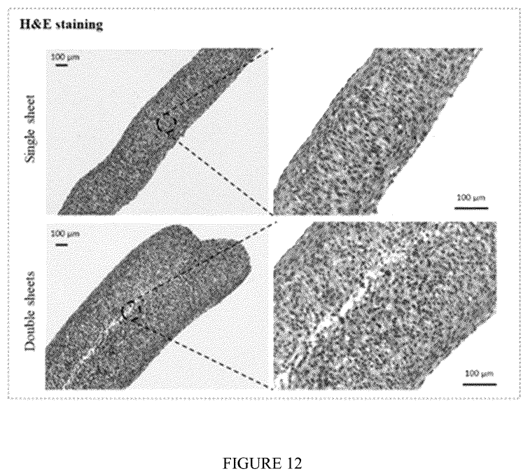

Histological assessment: To more precisely study the microstructure and cellular distribution inside the cell sheet constructs for both single-layer and double-layer stacked constructs, histological staining was performed. Briefly, samples were fixed in 4% PFA for 30 minutes. Then stepwise dehydration by immersing them in the gradually concentrated ethanol in DI water was performed. Next, the dehydrated constructs were undergone paraffin embedding, followed by sectioning to have a cross-sectional view of constructs, and performed staining with hematoxylin and eosin (H&E). The images were captured using inverted brightfield microscopy (EVOS microscopy) after 2 days of single-sheet formation and 1 day of performing the stacking procedure. As FIG. 12 shows, the cross-sectional images of H&E stained single-layered cell sheet after 2 days of harvesting indicates the densely packed cells formed the constructs with a thickness of 250-280 μm. Besides, the cross-sectional images of double-layer stacked constructs after 1 day of stacking show some degree of the layer's integrity at their interface, while the total fusion is not occurred yet (FIG. 12). Even though the full integration may need more time, the layers attachment can be attributed to the primary cell-cell, cell-ECM, and ECM-ECM attachment between these two separated sheets.

### Example 2

Developed here is a simple biofabrication technique where it is shown that use of ultralow adhesion surfaces of both solid and liquid can produce cell sheets if sufficient density of cells is simultaneously seeded on it. Pristine Polydimethylsiloxane (PDMS) (crosslinked solid or liquid) is used as the low surface energy and low adhesion surface and it is demonstrated that seeding cells at a density that is close to a monolayer density (>0.7 monolayer density) or greater, leads to assembly of the cells into an integrated sheet like tissue that is automatically detached from the surface but maintains its sheet like structure that is several cell layers in thickness after shrinkage. Untreated PDMS molds do not provide any anchoring locations for the cells to attach. When adherent cells are seeded at densities where sufficient number of cells are in close proximity, it preferentially promotes formation of cell-cell connections. When these connections happen simultaneously a sheet structure is formed in as little as 4-6 h instead of spheroids or aggregates as normally observed at lower seeding densities. The method is simple, fast and inexpensive without the need for any surface modification, use of specific equipment, or application of external stimuli. Cell sheets formed with this technique are mechanically robust and can tolerate robust manipulation such as transferring to another containers as well as stacking, and assembly to form thicker and more complex structures using tweezers. Furthermore, cell sheets were formed out of a variety of cell types in different shapes, sizes, and patterns which enables coculturing of different cell types and make more physiological-relevant tissue constructs. Also demonstrated is a scalable pick and place technique to assemble more complex patterns of cell sheets. Also shown is the biocompatibility of UC-MSC cell sheets after transplantation into the full-thickness burn porcine model over 40 days of wound healing in vivo study.

### Materials and Methods

### PDMS Mold Fabrication

The process of cell sheet formation begins with the fabrication of pristine Poly dimethyl siloxane (PDMS) molds with proper shapes and sizes that represent the ultimate shape and proportional size of cultivated cell sheets. To this purpose, negative shapes of desired features were designed using Solidworks® software, and then Acrylonitrile Butadiene Styrene (ABS) master molds were 3D-printed using a stereolithography-based Ultimaker 3D printer. Next, a mixture of PDMS (Dow Corning Sylgard 184) and its curing agent with the ratio of 10:1 was made and degassed under vacuum condition for 1 h. The degassed mixture was cast onto 3D-printed master molds and incubated at 80° C. for 2 h to solidify and were peeled off. Finally, they were sterilized by heating them up to 120° C. for 10 minutes and sprayed 70% ethanol all over the surfaces and let them dry out under the Bio-Safety Cabinet (BSC) and use for cell culture purposes.

### Cell Culture

Various cell lines were cultured and grown in regular polystyrene tissue culture plates prior to detachment and used to form scaffold-free cell sheets. As a primary cell source in this study, green fluorescent protein (gfp) NIH-3t3 fibroblast cell lines (Cell biolabs, Cat #: AKR-214) were grown in their proper culture medium, Dulbecco's Modified Eagle Medium (DMEM, high glucose, Gibco™ Cat #: 11965092) supplemented with 10% V/V Fetal Bovine Serum (FBS, Gibco™ Cat #: 12483020), 1% V/V penicillin-streptomycin (10K U/ml, Gibco™ Cat #: 15140122). Other alternatives cell lines such as C2C12 mouse myoblasts, 3T3-L1 mouse preadipocytes, human primary skeletal muscle cells (HSKMCs), SaOs-2 human osteosarcoma cell line, and BeWo human placenta choriocarcinoma cells were also grown in their specific culture medium. C2C12 cells were grown in DMEM (high glucose, Gibco™ Cat #: 11965092), supplemented with 10% V/V heat-inactivated FBS (HI-FBS, Canadian origin, Gibco™ Cat #: 12484028), and 1% V/V penicillin-streptomycin. 3T3-L1 cells were cultured in DMEM+GlutaMAX (low glucose, Gibco™ Cat #: 10567014) with 10% V/V HI-FBS and 1% V/V penicillin-streptomycin. HSKMCs were grown in their ready-to-use growth medium (PromoCell Cat #: C-23060) and differentiated using the ready-to-use differentiation medium (PromoCell Cat #: C-23061). SaOs-2 cells were cultured in McCoy's medium (5A modified, Gibco™ Cat #: 16600082) supplemented with 15% FBS, and 1% penicillin-streptomycin, and BeWo cells were grown in Ham's F-12K (Kaighn's) medium (Gibco™ Cat #: 21127022) containing 10% FBS and 1% penicillin-streptomycin. In addition to these cell sources, red fluorescent protein (rfp) tagged Human umbilical vein endothelial cells (HUVECs), were used to make perivascularized cell sheets. rfp-HUVECs were grown in Endothelial Cell Growth Medium 2 (EGM2) (PromoCell Cat #: C-22011). Primary bovine satellite cells (Opo-Moo-M17) were purchased from Opo Bio company, New Zealand. These cells were grown in their growth medium containing Ham's F-10 Nutrient Mix (Gibco™ Cat #: 11550043), 20% FBS, 0.625 μg/ml amphotericin B ((Gibco™ Cat #: 15290026), 1% penicillin-streptomycin, and 5 ng/ml bFGF Recombinant Protein (human heat stable, Gibco™ Cat #: PHG0367). Human burn-derived mesenchymal stem cells (BD-MSCs), human umbilical cord-derived mesenchymal stem cells (UC-MSCs), and primary human normal skin fibroblasts (NF) were kind gifts from Dr. Marc Jeschke's research group, David Braley Research Institute (DBRI), McMaster University. BD-MSCs and NF were cultured in CTS™ KnockOut™ DMEM/F12 medium (Gibco™ Cat #: A1370801) with 10% FBS and 1% Antibiotic-Antimycotic (Ab/Am) (Gibco™ Cat #: 15240112). UC-MSCs were also grown in CTS™ KnockOut™ DMEM/F12 medium (Gibco™ Cat #: A1370801) supplemented with 10% FBS, 1% Ab/Am, and 1% MEM Non-Essential Amino Acids Solution (NEAA) (Gibco™ Cat #: 11140076).

### Cell Sheet Formation

Scaffold-free cell sheet formation starts with detachment of individual GFP-3t3 cells from the tissue culture plates using trypsin when it reaches 80-90% of confluency. Next, to optimize the required number of cells to form a continuous and thin sheet, cells were counted and various numbers of them ranging from 0.3×10⁶ to 2×10⁶ cells were aliquoted, spined down, and resuspended in 300 μL of growth medium. Then, they were subsequently poured into circular PDMS molds with 1 cm diameter and incubated at 37° C. and 5% CO₂. The same procedure was employed to make cell sheets out of the other six additional cell types mentioned previously. Brightfield imaging of cell sheets was performed after 1 day of culturing cells in PDMS molds using stereo microscope (Infinity Optical Systems) to investigate the critical cell density for making well-integrated flat sheets. Brightfield images were also taken after 6 h, 1, 2, and 4 days to measure the shrinkage of cell sheets over the relatively long-term culture period.

### Scalability of Cell Sheet Manufacturing and Assembly to Form Complex Structures

Three different strategies were employed to scale up the cell sheet biofabrication technique, including making larger sizes of cell sheets, cell sheet staking, and assembly. Various sizes of circular PDMS molds (D=2, 3, and 8 cm) and different PDMS mold shapes such as square, cross, star, and dumbbell were made following the previous fabrication process. Then, based on their surface area, different cell numbers were loaded into them to maintain the same cell density followed by 1 day of incubation at 37° C. and 5% CO₂. Thicker tissue-like constructs were made by stacking either two (double-layered constructs) or three (triple-layered constructs) individual cell sheets on top of each other and capability of adhesion of cell sheets to each other was also investigated. The stacking procedure was performed in a regular 24-well plate where 2 ml of culture medium was added to the wells and cell sheets were stacked simply by picking them using a tweezer and piling them up on each other in a manual pick and place operation. Such operations can be automated using robotic handlers. Then, nearly all the medium was aspirated to let the sheets settle down and increase their contact area due to their weight for 15 minutes and then 2 ml fresh growth medium was gently added to them to prevent disruption of stacked sheets. The images of samples were taken immediately after stacking, one, and three days after keeping them in culture using stereo microscope and their diameter was calculated using ImageJ software at this time points to study the effect of number of cell sheets on the shrinkage pattern. More complicated macrostructures were also formed by cell sheet assembly. This method has been used to assemble larger sheets from smaller sheets. The individual dumbbell-shaped cell sheets were formed as the unit block of defined shape and assembled into a larger structure by using a tweezer in abundant amount of medium to connect these unit blocks with each other by overlapping the end units. After 15 minutes, the unit parts settled down and proper volume of medium was added to them to supply enough nutrients. After 1 day, their fusion and integrity were tested.

### Patterning and Making Multicellular Cell Sheets

Four strategies were used in this study to investigate the possibility of patterning and making cell sheets with multiple cells in culture. NIH-3t3 cells were grown in their culture medium until 90% confluency and then stained with DiO and DiL fluorescent cell trackers (Thermofisher) for ease of imaging purposes. Next, the cells were trypsinized and different strategies were employed to form heterogenous cell sheets. First, a 3D-printed Polylactic acid (PLA) external insert with separation wall thickness of 600 μm were fabricated to separate the PDMS chamber area into two distinct sections. After placing the external insert to the circular PDMS mold (D=1 cm), 0.75×10⁶ DiO-stained and DiL-stained cells were deposited to the different sides of the insert, subsequently. The inserts were then removed after 15 minutes while cells were settled down due to the gravity force. The second technique is to subsequently add different cellular suspensions to a confined PDMS mold. The dumbbell-shape PDMS structure was chosen as the constrained shape of the mold here, and DiO/DiL-stained 3t3 fibroblast cells were poured into each section at intervals of 15 minutes and kept incubated at 37° C. for 1 day.

Additionally, a bilayer construct was formed by serial addition of DiL and DiO-stained fibroblasts to a single PDMS mold one after another. Briefly, 1.5×10⁶ DiL-stained 3t3 cells were first added to the PDMS mold, followed by 30 minutes incubation at 37° C. and 5% CO₂. The cells settled to the bottom of the well and assembled within the 30 mins. Then the excess volume of culture medium was slowly aspirated and the same number of DiO-stained cells were gently added to the PDMS mold on top of the previous cellular layer and were incubated to let the cells to complete their self-assembly and form cell-cell connection. Besides, a mixed cell suspension contained both DiO and DiL-stained fibroblasts with each cellular concentration of 0.75×10⁶ cells was made and added to the PDMS mold to form a mixed cell sheet.

### Formation of Perivascular Cell Sheet

To make perivascular cell sheets, gfp-3t3 fibroblasts were cocultured with rfp-HUVECs endothelial cells. These cells were grown in their proper culture medium up to 90% confluency and then trypsinized and resuspend in a 1:4 ratio of HUVECs to gfp-3t3 in fibroblast growth medium. The mixture was then poured into the 1 cm diameter PDMS mold and kept incubated at 37° C. and 5% CO₂ for 4 days and the media was refreshed every day. The microstructure evolution of the construct was monitored during the culture period using Zeiss inverted fluorescent microscopy.

### Microstructure Evaluation

Alexa Fluor™ 488 Phalloidin, and DAPI (4′,6-diamidino-2-phenylindole), live/dead, and histological assessments were performed to study the microstructure and cellular distribution in cell sheets. NIH/3t3 cell sheets were fixed after one day of formation by immersion in 4% paraformaldehyde solution for 30 minutes and then washed three times with Phosphate-Buffered Saline (PBS). Then, the staining solution were prepared by dilution of Alexa Fluor™ 488 Phalloidin stock solution (300 units dissolved in 1.5 ml methanol) and DAPI stock solution (10 mg/mL in DI water) in 0.1% Triton X-100 in PBS with the ratios of 1:1000 and 1:2000, respectively, to morphologically assess the microstructure of cell sheet. Next, 500 μL of staining solution was added to the cell sheets and kept in RT for 1 hr. Confocal microscopy was used to image the sheets in 3D. To perform live/dead staining on cell sheets after one day of formation, Viability/Cytotoxicity Kit (ThermoFisher) were used and Calcien-AM and Ethidium homodimer were diluted in fibroblast growth media with 1:2000 and 1:500 ratios, respectively. Then, the staining solution was added to the cell sheets, and incubated at 37° C. for 30 minutes. After washing the dyes with PBS, the live/dead imaging was captured using an inverted fluorescent microscope (Olympus) and using proper filters of TEXRED and FITC. For histological assessments, fixed samples were dehydrated gradually and embedded in paraffin, followed by sectioning to get the cross-sectional view of them. Then, staining was performed using hematoxylin and cosin (H&E) as well as Picrosirius Red (PSR), and images were captured using inverted microscopy with proper filters (EVOS microscopy).

### Cellular Behavior Assessment

Cellular behavior for fibroblast cells in monolayer format (2D culture), and self-assembled cell sheet format (single, double, and triple layers) was assessed using Alamar blue assay (ABA) to measure the metabolic activity of cells, Pierce BCA Protein assay for total protein content measurement, and 1-Step qRT-PCR (quantitative reverse transcription polymerase chain reaction) to evaluate integrin and β-cadherin expression of cells, all using ThermoFisher kits.

For ABA, a 10% V/V Alamar blue solution in fibroblast growth medium was prepared and 500 μL of the solution was added to the samples followed by 90 minutes incubation in dark at 37° C. then, the fluorescent intensity of three 100 μL aliquots for each sample were read using the plate reader (Tecan Infinite M200 Pro) at excitation/emission of 560/590 nm. The Alamar blue containing solution was used as negative control while the 2D monolayer of cells was considered as the positive control (n=3 with triplicate reading).

Total protein content of cell sheets was measured starting by digesting the sheets and 2D monolayer (control group) using 500 μL of digestion solution contained 2 mg/ml of collagenase/dispase (Sigma-Aldrich, cat #10269638001) in PBS for 2 h at room temperature (RT). Then, the content of each well was mixed vigorously to break the sheets and make a homogenous mixture. 100 μL of digested solution was then lysed in 100 μL of 0.5% V/V Triton X-100 in PBS solution for 15 min at RT. Next, by following the instruction of Pierce™ BCA Protein Assay Kit (Thermofisher, cat #23227), 25 μL of each sample replicate were pipetted into a 96-multi well plate and then 200 μL of BCA working reagent (50:1 ratio mixture of reagent A:B) were added to each well and mixed thoroughly using a shaker for 30 sec. Then, samples were incubated at 37° C. for 30 min followed by cooling down to RT for 5 min and measuring the absorbance at 562 nm using the plate reader (Tecan Infinite M200 Pro). 2D monolayer was considered as positive control and mixture of digestion and lysing solution was used as negative control (n=3 with triplicate reading).

Quantitative reverse transcription polymerase chain reaction (RT-qPCR) was performed on self-assembled single cell sheet layer of 3t3 fibroblasts and were compared to 3t3 cells plated at the same cell density in 2D culture plate (control group). E-cadherin (cell-cell adhesion indicator) and β1-integrin (Cell-ECM adhesion indicator) and β-actin (housekeeping gene) were chosen as the target genes (table 1). Samples were digested using 500 μL of 2 mg/ml collagenase/dispase for 2 h at room temperature. Then, cells were counted and digested solutions were gently spined down to remove the collagenase/dispase, followed by resuspension in 50 μL chilled PBS per 10⁵ cells. Again, gently centrifuge to pellet the cells and aspirate as much PBS as possible. Then, the pellet resuspended in 5 μL chilled PBS per 10⁵ cells and 5 μL of cells for each group of samples were distributed to 96-well PCR plate (n=3 for each sample). Next, 50 μL of lysis solution (provided in the kit) was added to them followed by 5 minutes of incubation at RT. After 2 minutes of stop solution (5 L) addition, the cells lysates were ready to run the RT-qPCR test. A master mix was then prepared using the reagents provided in the Cells-to-CT™ 1-step PowerSYBR® Green kit (ThermoFisher) and gene-specific primers following the instruction of kit for 20 μL reaction containing 2 μL of cell lysate. The reactions were run in a RT-qPCR machine for 3 samples per each primer set or nuclease-free water as no template control (NTC). The ΔΔCt values for each primer were normalized to the average value of housekeeping gene primer Ct value using Bio-Rad CFX manager software.

TABLE 1
Primer sequences used for qPCR (5′ to 3′)

| Target | | |
|---|---|---|
| Gene | Forward | Reverse |
| E- | AACCCAAGCACGTAT | ACTGCTGGTCAGGATCGT |
| cadherin | CAGGG (SEQ ID | TG (SEQ ID NO: 2) |
| | NO: 1) | |
| β1- | AATGTGTTCAGTGCA | TTGGGATGATGTCGGGAC |
| integrin | GAGC (SEQ ID | (SEQ ID NO: 4) |
| | NO: 3) | |
| B-actin | GGCTGTATTCCCCTC | CCAGTTGGTAACAATGCC |
| | CATCG (SEQ ID | ATGT (SEQ ID NO: |
| | NO: 5) | 6) |

### In Vivo Full-Thickness Burn Porcine Model and Wound Healing Study

### Porcine Full-Thickness Burn Model

A validated porcine full-thickness burn model was employed as previously described (Eylert et al., 2021). This study was approved and performed in accordance with the guidelines and regulations of the McMaster University's Animal Research Ethics Board (AREB) following the approved animal use protocol AUP #(22-08-29). Briefly, Yorkshire pigs were chosen due to the similar anatomic and physiologic skin characteristics and comparable pigmentation to human skin. Two weeks after acclimatization in an animal facility multiple 5×5 cm wounds were introduced on the dorsum of a female Yorkshire pig with a weight of 35 kg and dorsal length of 60 cm, following the standardized protocol described below under general anesthesia with 5%/L O₂ isoflurane and analgesia (subcutaneous 0.05 mg/kg Buprenorphine, 0.2 mg/kg Ketamine, combined with 0.5-1.0 mg Atropine). The full-thickness burn injuries were induced by a 5×5 cm heated aluminum device (200° C.) (t=day-2). A 3 cm interspace was maintained between adjacent wounds. Further analgesia (2-4 mg/kg Tramadol every 8 h orally) was administered as per the veterinarian's recommendations.

Full-thickness burn tissue excision and hemostasis were performed 48 hours post-burn down to the healthy muscle fascia on the grafting day (t=day 0). Wounds were treated with a 2.5×2.5 cm prepared UC-MSCs cell sheet, UC-MSC-cellularized Integra® Meshed Dermal Regeneration Template (Integra® MDRT, Integra LifeSciences), and the acellular control (Integra® MDRT alone). A further control wound was left ungrafted, termed as burn alone group. UC-MSC-cellularized Integra® MDRT, at a low dose seeding density of 10,000 cells/cm² (also referred to as Integra 10K UC-MSC group), was produced following the same procedure as described previously (Eylert et al., 2021). All scaffolds were fixed via skin staplers on the wound corners except UC-MSCs cell sheet due to its immediate integration to the wound site after grafting. Regular wound dressing changes (2-3 times/week) were performed at determined timepoints. The wounds were covered with paraffin non adherent dressing and wet to dry sterile gauze dressings that were kept in place by the adhesive, breathable film dressing, a costume-made porcine jacket (Lomir Biomedical Inc.) and an elastic spandex tube (Weaver Leather, LLC). During the study, the veterinarian and veterinarian technicians monitored the procedure and wellbeing with the standard safety and health checks. The investigated animal maintained its health during the entire study period without any adverse events. On day 40 the pig was euthanized by intravenous injection of 100 mg/kg Pentobarbital.

### Wound Healing Assessment

During the regular dressing changes, wound healing and epithelialization were assessed by wound scoring and gross images acquired from a mirrorless digital camera (Sony a7R II). The wounds were closely monitored for unfavorable outcomes including infection, inflammation, and bleeding. Wounds were photographed at each dressing change for gross appearance and wound closure analysis. Images were analyzed using ImageJ software to determine epithelialization and contraction. Scar quality on day 40 was independently and blindly assessed by three surgeons using the Vancouver Scar Scale, evaluating the vascularity, pigmentation, pliability, and height of the scars.

### Statistical Analysis

All the reported data are presented as Mean±Standard Deviation (SD), and groups of samples with p-value of less than 0.05 were considered as significant statistical different by performing student t test using Excel.

### Results and Discussion

### Cell Sheet Formation Process

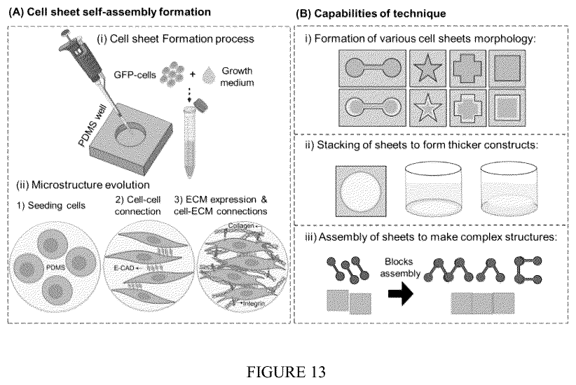

Adherent cells naturally adhere either to the substrate or to other cells. Using this principle, a new biofabrication method has been developed to produce large sized cell sheets. Typically, when cells seeded at low densities on surfaces with low surface energy, the distribution of cells is sparse and therefore local cell aggregates (spheroidal structures) are formed (FIG. 13A). This is due to the preference of cell-cell adhesion over cell-substrate adhesion in these substrates. However, it is demonstrated here that seeding at near confluent densities can result in simultaneous connection between the cells over a large area and thus lead to formation of cell sheets instead of spheroids (FIG. 13A). The cell sheet formation process begins by filling the Polydimethylsiloxane (PDMS) wells with a cell suspension at the desired cellular density which is followed by the self-assembly of the cells during incubation at 37° C. and 5% CO₂ for a short period (FIG. 13A). The self-assembly procedure initiates within 15 minutes after seeding the cells into the PDMS well, as the cells settle at the well's bottom due to gravitational force. As the cells cover the PDMS surface, their lateral movement becomes more restricted, and self-assembly commences. Pristine PDMS molds have low surface energy and do not provide any anchoring locations for cells to attach. Thus, when adherent cells are seeded at densities that ensure a sufficient number of cells in close proximity, it leads to the formation of cell-cell connections rather than cell-substrate adhesion. As cell-cell adhesions strengthen, a planar self-assembled cell sheet forms instead of spheroids or aggregates.

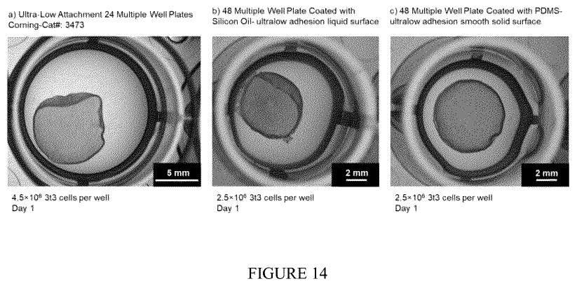

It is also shown that different non-adherent surfaces in either solid or liquid formats such as ultra-low adhesion tissue culture plates, and liquid silicone oil can be utilized to culture high cellular density. The results indicated that all low surface energy substrates support cell sheet formation (FIG. 14), while the same procedure implemented on conventional adherent tissue culture plates led to attachment of all the cells to the substrate and no cell sheet formation. These self-assembled cell sheets can withstand handling procedures, such as using tweezers to transport it to a larger container for better nutrient access. Using molds of different shapes, the cell sheets with those shapes can be easily formed (FIG. 13A, i). Also, thicker layers and more complicated tissue-like constructs can be assembled by stacking individual sheets on top of each other (FIG. 13A, ii, and iii).

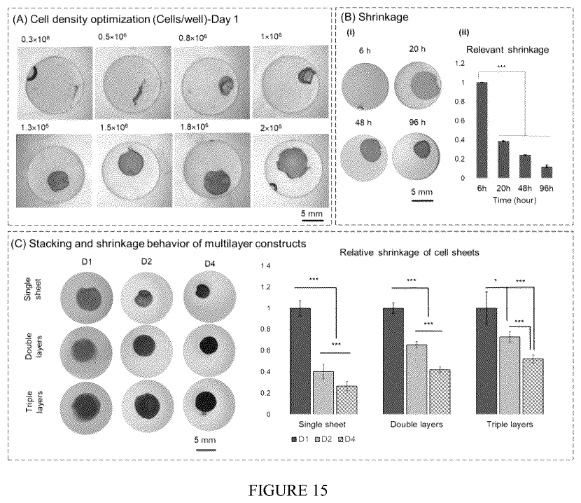

In order to determine the critical cell density required for forming a well-integrated and thin cell sheet, cell suspensions of varying densities ranging from 0.3×10⁶ to 2×10⁶ cells/well were loaded into the circular PDMS molds (Diameter=1 cm). After incubating for one day at 37° C. and 5% CO₂, the samples were imaged. As shown in FIG. 15A, lower seeding densities of cells below 0.8×10⁶ cells/well resulted in scattered aggregation of the cells into spheroids or multispheroidal assemblages. When the seeding density was increased to greater than 0.8×10⁶ cells/well a distinct formation of sheet-like contiguous construct was seen. This construct was not attached to the substrate and can be easily removed. However, at intermediate cellular densities (0.8-1×10⁶ cells/well) to the molds, the sheet formed was not as robust and resulted in rolled up edges and self folding upon itself during the formation process. By increasing the cell density to above 1.3×10⁶ cells/well, continuous and well-integrated sheets were produced that remained flat and circular, exhibiting sufficient mechanical robustness for manipulation. It is also worth mentioning that by increasing the cellular density over 1.3×10⁶ cells/well, cells were self-assembled to the larger and thicker cell sheets in the same PDMS well size. Calculations show that approximately 1.2×10⁶ cells are needed to cover the surface of a 1 cm diameter PDMS circular mold with trypsinized cells to form a monolayer. Therefore, the findings indicate that for creating planar cell sheets, the critical cell density must be more than 0.7 times the cell density needed for monolayer coverage. All together due to the suitable mechanical robustness as well as consistency of cell sheet formation, 1.5×10⁶ cells per 1 cm diameter circular PDMS mold (˜1.9×10⁶ cells/cm²) was chosen as the optimal cell density. The simultaneous seeding of cells at high density results in the cells encountering several cell-cell interactions with their neighbours forming linkages. This combined with the lack of adhesion motifs on the substrate leads to formation of floating cell sheets that are not adhered to the substrate. At low seeding densities, the distribution of the cells is sparse resulting in local aggregation which does not have sufficient size to remain as sheets and curl up to for spheroidal aggregates. However, without being bound by theory, above the critical density, the traction forces that are exhibited due to simultaneous cell-cell contacts at various locations across the cell sheets balance each other and ensure that a single contiguous cell sheet is formed and remains planar.

The contraction of a single-layer cell sheet was characterized by seeding 1.5×10⁶ cells into a circular PDMS mold (D=1 cm). Images were captured at 6 h, 20 h, 48 h, and 96 h intervals to measure their diameter using ImageJ. It was observed that the optimal timing for 3T3 fibroblast cells to form a robust self-assembled cell sheet can be as short as 4-6 h, depending on the initial cell density seeded into the PDMS well. For 1.5×10⁶ cells, it took about 6 h to form a mechanically robust cell sheet. The contraction behavior of single layer cell sheet was monitored over a 4-day culture period. As FIG. 15B indicates, a single-layer cell sheet exhibited a 61.57±3.9% contraction after 1 day of incubation. However, the rate of contraction decelerated after 48 h and 96 h of incubation. The relative shrinkage of the cell sheet on day 2 was 59.92±1.44% from day 1, and then it subsequently shrank by 33.8±0.89% on day 4 from its size on day 2. Additionally, cell sheets were stacked atop each other to form thicker, tissue-like constructs. The contraction behavior of multilayered constructs (double and triple-layered constructs) was studied and compared to single-layer cell sheets over a 4-day experimental period (FIG. 15C). Images were taken immediately after stacking (D1), 1 day post-stacking (D2), and 3 days post-stacking (D4) following incubation of the stacked cell sheets at 37° C. and 5% CO₂. Results revealed that single-layer cell sheets contracted more than multilayer constructs. As the number of cell sheet layers increased, the extent of contraction diminished. Single sheets displayed a 59.92±6.59% contraction after 1 day, while the diameters of double-layered and triple-layered constructs reduced only by 34.80±3.04% and 27.25±5.09%, respectively. Similar findings were observed after 3 days of culture: the diameter of a single sheet contracted to 73.48±4.31% of its initial value, whereas double and triple-layered constructs shrank by 58.09±2.70% and 47.95±4.06%, respectively.

Once trypsinized cells of sufficient density are seeded into a non-adherent PDMS mold, adjacent cells immediately begin to form cell-cell connections, as there are no suitable anchorage sites on the surface for cell-substrate attachment. As a result of the lack of surface adhesion forces and the strengthening of cell-cell connections, traction forces are exerted and cell sheet shrinkage occurs, leading to the formation of thicker, dense 3D tissue-like constructs. During the first few hours of assembly, adjacent cell-cell connections begin to form but may not be sufficiently dense enough to exert significant traction forces which can lead to macroscopic contraction. However, after a few hours sufficient number of cell-cell connections form that facilitate traction forces to increase and cause the shrinkage of the cell sheet toward the center of the construct. The traction force is radially applied in all directions towards the center of the sheet, maintaining a flat profile and is highest during the first day after seeding and decreases significantly on subsequent days. Any imbalance in the traction forces can lead to out of plane folding of the sheets and sheet agglomeration over extended culture times (Takezawa et al., 1990). It was observed that at lower cellular densities (0.8-1×10⁶ cells per 1 cm diameter) the sheet spontaneously curls up at the edges. Without being bound by theory, this is probably due to lower cell density at these areas and uneven distribution of cells which result in unbalanced traction forces at the edges. When the cell density is increased, the seeding is more uniform and in some cases multilayered which produces thicker sheets that balance the traction forces. The cell sheets generated at these cell densities are mechanically robust and can easily handle multiple cycles of pipetting and handling using tweezers. Furthermore, the production of extracellular matrix (ECM) by cells leads to strengthened cell-ECM connections, compensating for the contractile forces generated by the cellular cytoskeleton. Thus, sheets remained flat and preserve their initial shape. The individual cell sheets formed are naturally adhesive to other natural ECM like collagen. Therefore, when cell sheets are stacked to form multilayered constructs, the adhesive forces between the ECM on different layers form normal anchorages between the cell sheets and prevent subsequent contraction. The degree of contraction observed in mutilayered sheets are substantially less than that observed in single layer sheets.

One interesting aspect of the technique described herein is that the natural tendency of the adherent cells to form cell-cell connections advantageously by using low surface energy substrates that promote the cell sheet formation is used. No harsh treatments such as thermal shock, mechanical scraping, enzymatic treatments, or chemical shock (by changing the pH of culture medium) used by others which can potentially stress the cells and introduce protective mechanisms related to stress exposure are used here. Furthermore, the method is low cost, rapid and simple to implement without requirement for any specialized equipment and produces cell sheets that are mechanically robust, maintain their flatness, and are scalable to large dimensions.

### Microstructure Evaluation of Cell Sheets

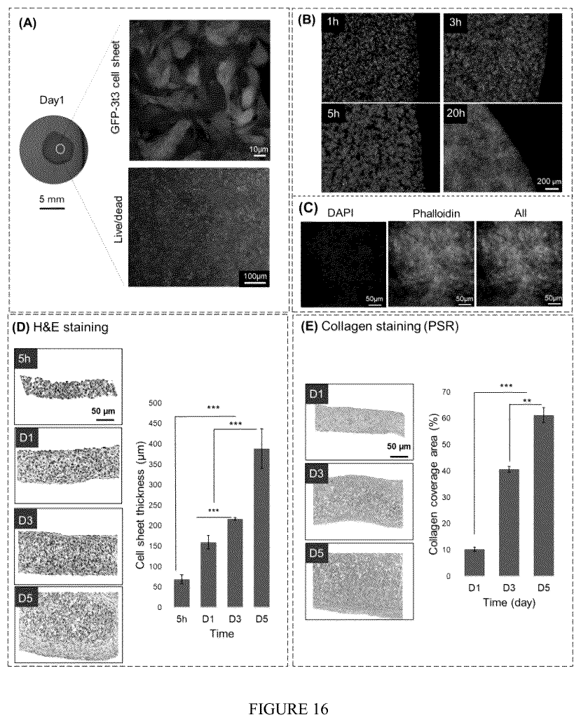

Various types of staining were performed to evaluate the effect of self-assembly procedure on cellular viability and microstructure, images were taken using fluorescent and brightfield microscopy, and analyzed using ImageJ. First, the morphology of cells in cell sheet format after 1 day of culture were studied using 1.5×10⁶ GFP-3t3 cells in culture. As FIG. 16A shows, cells are tightly packed and adhered to each other to make strong cell-cell connections. To get better insights on how cells are distributed inside the sheets, Phalloidin and DAPI staining were performed on NIH-3t3 fibroblasts cell sheet with 1.5×10⁶ cells/well initial cell density after 1 day. The results (FIG. 16C) show a densely packed (5.52×10⁵ cells/cm²) microstructure which is consistent with the morphological assessment of GFP-3t3 cells in cell sheet format. Noticeably the cellular nuclei are distributed all over the surface homogenously at close proximity, while actin filaments of the cellular cytoskeleton formed well-integrated and interconnected network which would cause the cell sheet to shrink and make mechanically robust construct. Moreover, live/dead staining of single layer cell sheet after 1 day of incubation (FIG. 16A) indicates that promoting self-assembly of cells to form planar cell sheets does not have adverse effect on cell viability as very few dead cells were present in the construct.

In order to determine the optimal timing for cells to adhere to each other and make a continuous and robust cell sheet, 1.5×10⁶ GFP-3t3 cells were seeded into the 1 cm circular PDMS mold and their microstructure evolution were monitored using fluorescent microscopy. Images were captured after 1 h, 3 h, 5 h, and 20 h of incubation. As shown in FIG. 3B, after 1 hr of seeding the cells into the mold, they are fully settled on the surface, but microscopic void spaces exist between them. At 3 hrs, the gaps between cells have diminished, and some degree of cell-cell connections are noticeable in the microstructure. However, these connections were not yet robust enough to make a contiguous cell sheet which can withstand mechanical handling. Examining the micrographs of the cell sheet after 5 h of incubation reveals that while some void spaces persist, a more interconnected, colony-like structure emerges, indicative of strengthening cell-cell connections between adjacent cells. At this stage, the cell sheet, although porous, can be removed from the PDMS mold and transferred to another container, as the cell-cell connections have achieved sufficient strength for mechanical handling. Cell sheet constructs continued to be incubated and were imaged after 20 h. The results showed a significant reduction in porosity, with the strengthening of cellular adhesion leading to shrinkage and the formation of multilayer constructs.

Images of the cross-section of H&E-stained single-layer cell sheets at various time points over a 5-day culture period were captured using inverted brightfield microscopy, to further characterize the evolution of the microstructure of the constructs. FIG. 16D demonstrates that after 5 h of seeding 1.5×10⁶ cells into the non-adherent PDMS well (D=1 cm), self-assembly took place, resulting in a dense planar construct with an average thickness of ˜70 μm, comprising 5-6 layers of cells. Consistent with the shrinkage behavior observed in cell sheets over time, the cross-sectional images of the samples also displayed a significant increase in the thickness of the constructs. The thickness of a single sheet grew to ˜160 μm, ˜217 μm, and ˜390 μm after 1, 3, and 5 days of incubation, respectively. Interestingly, it is seen that the cells self-assemble into an alignment parallel to the surface at the top and bottom of the cell sheet while the central part does not have a clear orientation preference. This microstructure evolves over the increasing number of days in culture is the first demonstration of self organized orientation of cells in a self assembled cell sheet. Without being bound by theory, this phenomenon might due to stresses imposed on the cell sheets due to traction forces exerted by the cells and the equilibrium configuration that is achieved in such evolution. It also explains the reason why such cell sheets remain planar rather than fold into a spheroidal configuration. The lack of orientation at the center can be due to the induction of fibroblast quiescence, a result of reduced oxygen and nutrient availability in those regions (Marescal & Cheeseman, 2020). Additionally, collagen staining was performed on cell sheets after 1, 3, and 5 days of culture using the Picrosirius Red (PSR) staining kit to determine whether the cells were functional and capable of producing their own ECM in the cell sheet format. The results revealed that the fibroblast cells produced collagen fibers, which are the primary and most abundant components in the connective tissue's ECM (Murray et al., 2009). The amount of collagen progressively increased during the culture period, starting from 10% of cell sheet cross-section area coverage on day 1 and reaching 40% and 60% on days 3 and 5, respectively. Compared to other methods where cells are cultured on 2D plates for extended periods to ensure ECM production before delamination, the self-assembly technique described herein is faster (De Pieri et al., 2021; Qian et al., 2018). This is due to a notably increased collagen expression within a shorter experimental timeframe. For instance, a study has shown that rat dermal fibroblasts were cultured on temperature-responsive tissue culture plates for a week until they reached confluency. These were then delaminated at a lower temperature (20° C.) for 30 minutes to produce a cell sheet (Nozaki et al., 2008). ECM expression evaluation revealed that collagen expression was around 32-35%, which is roughly equivalent to the collagen content in our cell sheets by day 3. Consequently, it can be inferred that the self-assembly of cells into 3D constructs does not compromise cellular functionality. Notably, in this study, the self-assembled fibroblast cell sheets exhibited higher ECM expression than their 2D-cultured counterparts. Without being bound by theory, this enhanced expression might result from the more physiologically representative morphology and microenvironment experienced by cells in the self-assembled cell sheet format.

### Assessment of Cellular Behaviors in Cell Sheet

The self-assembly of cells to form cell sheets and the subsequent stacking of these sheets to create thicker constructs have the potential to alter certain cellular behaviors, such as metabolic activity and protein expression. To investigate this, the metabolic activity of cells and their total protein content in the 3D cell sheet format were measured. For a clearer understanding, a 2D monolayer of cells cultured on conventional tissue culture plates (of the same size as the PDMS mold) was used as the control group. The same assays were then performed on them to compare the results with the 3D cell sheets. These control samples were prepared by culturing 1.5×10⁶ 3T3 cells in the wells of a 24-well plate. The metabolic activity assay was conducted after 5 h and one day (D1) of seeding cells in both the PDMS wells and tissue culture plates. The test was also continued for an additional day (D2) to assess the metabolic activity of the cell sheets.

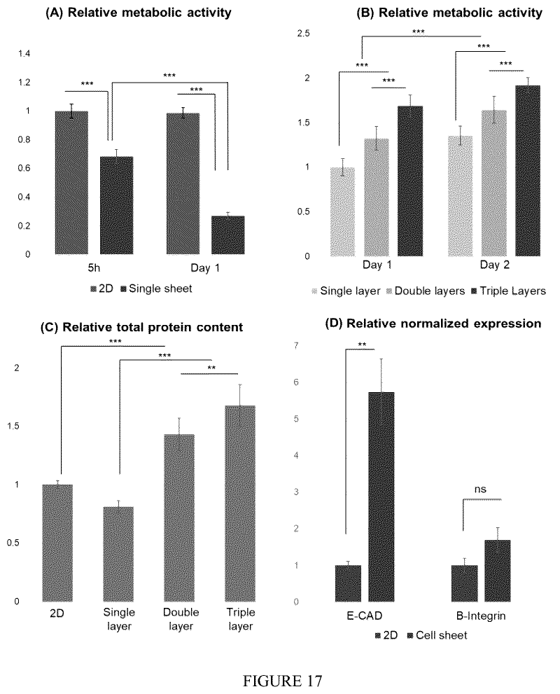

The results (FIG. 17A) showed that the metabolic activities of cells in the 2D monolayer group after 5 h and 1 day of culturing were identical to each other. However, significant reductions were observed in the single-layer cell sheets after 5 h (˜30%) and 1 day (˜70%) of culturing compared to the 2D monolayers. This reduction in metabolic activity might be attributed to differences in organization of cells in 2D cultures and 3D self-assembled constructs (Takezawa et al., 1993). In 2D culture, cells attach to the substrate and spread on the surface in a monolayer. In contrast, cells cultured on non-adherent substrates, such as PDMS wells, form 3D sheet like structures with multiple layers of cells. They initiate cell-cell connections to remain alive and functional and produce their own ECM, eventually forming cell-ECM connections, which are more physiologically relevant. The traction forces exerted contract the cell sheet and lead to a corresponding increase in the thickness as the cells are stacked on one another and become highly dense. The metabolic activity in the 3D cell sheets is directed towards greater production of ECM rather than cell proliferation as in the case of 2D. Furthermore, cells in a 2D monolayer have greater accessibility to nutrients and oxygen while in the case of 3D cell sheets there are gradients established as in in-vivo tissues (Takezawa et al., 1993). It explains the reasons for thicker multilayered constructs, where metabolic activity did not increase proportionally with the number of cell layers (FIG. 17B). Such challenges in nutrient and oxygen transport reduce the overall metabolic activity in double-layered and triple-layered constructs. It also suggests that excessively thick layers obtained by stacking more than 4 sheets can lead to formation of a necrotic core due to high cell density of these structures. Interestingly, after 2 days of culture, results for both single-sheet and multilayer constructs revealed a significant increase in cellular metabolic activities. This could be attributed to cellular proliferation within the constructs or changes in cellular morphology resulting from the secretion of ECM components and the increase in cell-ECM connections (FIG. 17B).

The total protein content of cell sheets (single and multilayer) was measured and compared to the 2D monolayer control group on day 1. The total protein content of the single-layer cell sheet was ˜80% of the 2D monolayer, increasing to ˜145% and ˜170% for double- and triple-layer constructs, respectively (FIG. 17C). The results indicate that the protein content of the single cell sheet is lower than the equivalent number of cells cultured as a 2D monolayer after one day. Cells seeded in 2D are provided with ample nutrients and oxygen due to their superior surface-to-volume ratio. As a result, their metabolic activity is higher than that of the 3D single sheet containing the same number of cells (FIG. 17A). The metabolic activity of the cells in 2D is directed more for cell proliferation as compared with ECM production while the lower metabolic activity in the 3D cell sheet is primarily directed towards greater ECM production which is less energy consuming and utilizes the available nutrients more effectively. Consequently, even though the metabolic activity of the 3D sheets is reduced by ˜70% from the 2D equivalent, the protein expression was only reduced by ˜20% due to greater ECM production in 3D cell sheets while most of the increase in protein content of the 2D monolayer can be attributed to proliferation. Without being bound by theory, this suggests that cells in 3D self-assembled cell sheet format might enter a quiescent state to express their own ECM and form cell-ECM interactions (Marescal & Cheeseman, 2020). Subsequently, these cells become primed for activation and rapidly express proteins vital for proliferation and other cellular behaviors. Based on the results of the live/dead assay, which displays a minimal number of dead cells after 1 day of culture (FIG. 16A), and the relatively high protein content compared to the 2D monolayer on day 1, the significant decline in metabolic activity of cells in the single cell sheet could arise from a shift to quiescence rather than necrosis. Thus, this self-assembly technique for creating cell sheets is neither toxic nor harmful to cells and supports the preservation of their functionality. The shift to quiescence also enables our method to produce more ECM within a shorter time frame as compared to other cell sheet formation methods.

Moreover, the protein content increased for double and triple-layer constructs (145% and 160% compared to the single layer, respectively) (FIG. 17C). However, for the triple-layered construct, the protein content's increase isn't directly proportional to the number of cell layers. This discrepancy might result of decreased oxygen and nutrient transport to the middle sheet from the periphery. Triple-layer constructs possess the greatest thickness among the groups, possibly slowing nutrient and oxygen transport to the center of the assembled construct, reducing the metabolic rate and thereby also lowering the production of ECM materials. These findings underscore the importance of incorporating blood vessels and microvasculature into even more thicker constructs to maintain their functionality.

In order to further investigate the microstructure of cell sheets, genetic assays were conducted to probe the expression of cellular adhesion proteins in cell sheet samples and compare them to the conventional 2D culture. As it is shown in FIG. 17D, E-cadherin, a cell-cell junction protein, are expressed 5.7-fold higher in cell sheet samples in comparison to 2D cultured cells, which explains the underlying mechanism of cellular self-assembly while cells are cultured in non-adherent substrates such as PDMS molds. Moreover, there is no significant difference between expression of β-Integrin, a cell-ECM or cell-substrate junction protein, in cell sheet and 2D cultured cells. These findings indicates that cell-cell adhesion is the main reason of cell sheet self-assembly while adequate number of cells are cultured in non-adherent PDMS mold. However, while self-assembly happened, cells begin to produce their own ECM components and cell-ECM adhesion occurred by expressing β-Integrin by cells to more strengthen their connections and compensating the traction forces applied by cell-cell connections which leads to the flatness of the cell sheets instead of formation of aggregates and spheroids during the experimental periods.

### Versatility of the Technique: Alternative Cell Types, Scalability, and Diverse Morphologies

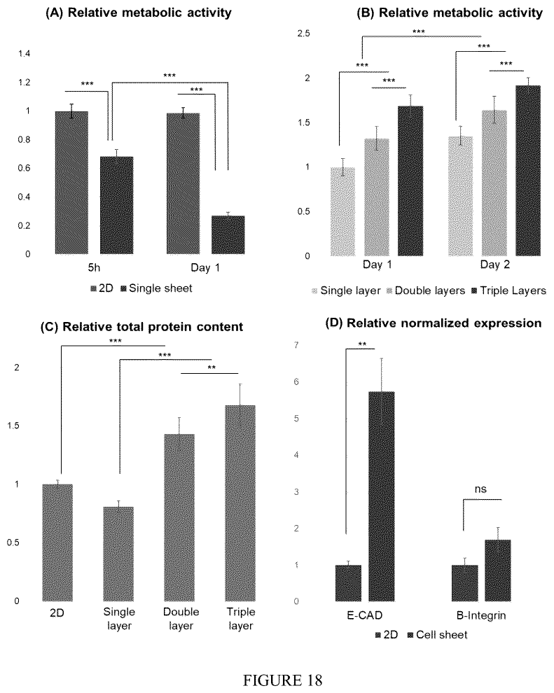

To demonstrate the versatility of the technique in creating diverse cell sheets, multiple types of adherent cells were seeded into a 1 cm diameter circular PDMS mold and incubated for 1 day. As depicted in FIG. 18A, our method successfully produced well-integrated and continuous cell sheets using primary human skeletal muscle cells (HSKMCs), mouse myoblasts (C2C12), mouse preadipocytes (3t3-L1), human cytotrophoblast placenta cells (BeWo), osteoblast-like cells derived from the human osteosarcoma cell line (Saos-2), human normal skin fibroblast (NF), human burn-derived mesenchymal stem cells (BD-MSCs), and human umbilical cord-derived mesenchymal stem cells (UC-MSCs). All these cell types exhibited self-assembly, resulting in the formation of robust and homogeneous sheets. In contrast, certain cell lines like human umbilical vein endothelial cells (HUVECs) and human corneal epithelial cells (HCECs) did not did not form a continuous cell sheet structure, indicating either a weaker cell-cell interaction or a non-sheet like self assembly. Nevertheless, this method was able to demonstrate a wider range of cell types that formed large sized continuous sheets as compared with a previously published method which used thermal or chemical shock (Shahin-Shamsabadi & Selvaganapathy, 2020). For example, sensitive cell types such as cytotrophoblasts and mesenchymal stem cells were demonstrated for the first time to form sheet like constructs. This expanded capability is particularly valuable for generating in vitro models of biological barriers, such as placental tissue as well as lab-grown meat cultivation and implantable tissue-like grafts.

Another crucial advantage of our technique is the ability to form large-area cell sheets. FIG. 18B illustrates the various sizes of cell sheet fabricated, ranging from approximately 0.5 cm to about 5 cm in diameter on day 1 post seeding cells into the PDMS mold. The required cell number to populate each surface was determined using 1.9×10⁶ cells/cm2 as the critical cell density. Larger cell sheets were successfully produced by adopting either increasing the PDMS mold size (FIG. 18B) or planar assembly of multiple smaller cell sheets at the sides of each other (FIG. 18D). The larger area sheets were also mechanically robust for handling using spatula or tweezers on day 1. These are advantageous for rapidly production of tissue-like constructs covering extensive areas as implantable tissue substitutes in biomedical applications and for creating actual-sized meat patties for cultivated meat applications. It is also noted that the rapidness of the technique in production of such large area sheets, could also enhance the therapeutic efficiency as well as significantly reduction in meat cultivation costs.

For creating more complicated structures, various shapes of PDMS molds (wells with different shapes), including square, cross, dumbbell, and star, were designed and fabricated. Cells seeded into these molds self-assembled to form sheets, maintaining their original shape even after shrinking, as shown in FIG. 18C. Hence, it can be concluded that the contraction force is applied uniformly in all directions towards the sheet's center. This ensures the cell sheets maintain their molded morphology upon the completion of the self-assembly process. In addition to these simple shapes, more complex macrostructures can also be crafted using cell sheet assembly. The individual cell sheets produced are inherently adhesive and can bind to other cell sheets or naturally produced ECM components. Using this characteristic, larger and more complex shapes can be assembled using elemental building blocks. As a demonstration, dumbbell shaped elemental building blocks were made and using a tweezer overlayed the end of adjacent blocks to assemble the letters of the word “MAC” thereby forming a larger and more complex design in 2D. Following assembly, the culture medium was completely removed from the well and the cell sheets allowed to integrate with each other for 30 mins. After this time the assembled and fused macrostructure was immersed in media for further culture. FIG. 18D shows the well integrated macrostructure shaped in the form of “MAC” after 1 day of incubation. These assembled units where monolithic, mechanically robust and can withstand multiple cycles of pipetting and tweezer handling without falling apart. This assembly method can be employed to assemble larger sheets from smaller ones or to wrap them around a sacrificial structure to achieve any desired 3D shape.

### Cell Sheet Patterning and Coculture Strategies

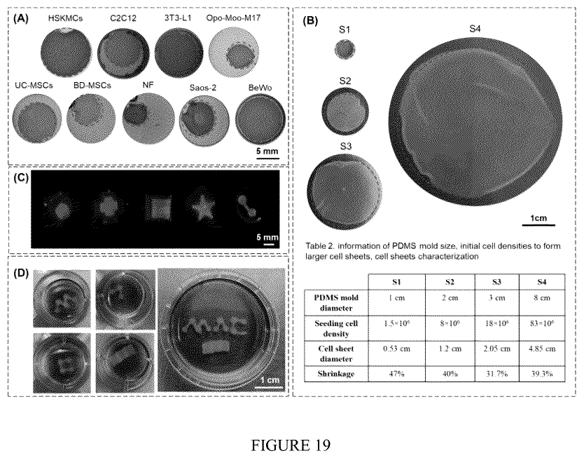

To replicate in vivo physiological conditions, the symbiotic interactions among various cell types within a specific tissue that is modelled should be considered. Additionally, the interactions of this tissue with other systems, such as the vascular and nervous systems, are also significant. Given the important influence of other cells and tissues on the functionality of our in vitro tissue models, suitability of this technique to mimic such interactions was assessed. This was done by developing several co-culturing and cellular patterning strategies. A co-culture system was established to create a bilayer cell sheet construct through the sequential deposition of pre-labeled cells into the PDMS well. For this, DiL-stained 3t3 cells were poured into the PDMS mold and incubated at 37° C. and 5% CO₂ for 30 minutes. This time was sufficient to allow the initial layer of cells to settle and cover the PDMS surface. Subsequently, the excess volume of culture medium was carefully removed without disturbing the integrity of the first layer. DiO-stained 3t3 cells were then gently added to the PDMS mold atop the previous layer. FIG. 19A-i illustrates the formation of two distinct and continuous cell layers, which after 20 hours of incubation, merged into a single co-cultured sheet. Incredibly, the confocal image of the sheet shows a clear delineation of the first layer and second layer of deposited cells. This result demonstrates that the first deposited cells form a contiguous layer within 30 mins and that subsequent deposition of cells on top of it will not disturb this base layer of cells. Alternatively, a mixed cell sheet can also be created by using an equal mixture of DiO and DiL-stained 3t3 fibroblasts, maintaining a 1:1 ratio (0.75×10⁶ of each pre-stained cell type) that are simultaneously seeded at the same time. This mixed cellular suspension was introduced into the PDMS mold and kept at 37° C. in a 5% CO₂ setting for 20 hours. FIG. 19A-ii shows the microstructure of this mixed co-culture cell sheet. As clearly visible in the cross-sectional micrographs, an interface is discernible between the top and bottom layers of the bilayer co-culture system, even though they are firmly adhered to one another. Conversely, in the mixed co-culture system, a uniform distribution of labeled cells can be observed throughout the cross-sectional view of the cell sheet.

To pattern cells in co-culture systems, two distinct strategies were developed. First, a suitable mold consisting of several regions to deposit bioinks and connected by thin narrow sections to limit the spread of the inks was used. As an example, a dumbbell shaped PDMS structure served as the mold which had two large cylindrical wells for cell seeding and which were connected by a narrow neck that serves to confine the cells to their respective regions. DiO/DiL-stained 3t3 fibroblast cells were added to each cylindrical section at 15-minute intervals. These bioinks did not spread to the other regions as they were confined to their respective halves due to surface tension. After 20 hours of incubation at 37° C. with 5% CO₂, a well-defined border between these two sections was observed. FIG. 19A-iii displays the macrostructure of this patterned sheet, which was constructed by the strategic deposition of cell suspensions. These cells integrated well, forming a singular monolithic unit by the end.

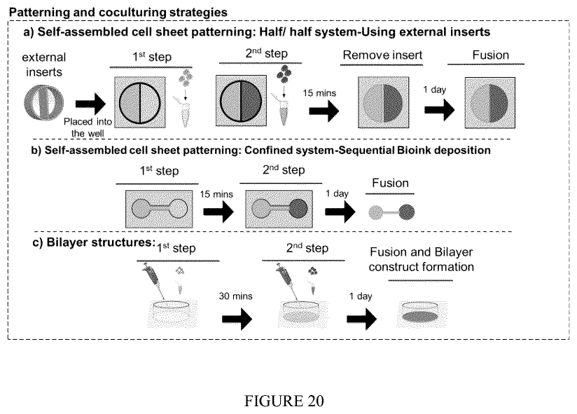

Alternatively, a temporary PLA external vertical insert with wall thickness of 600 μm was 3D-printed to partition the circular PDMS chamber area (D=1 cm) into two distinct sections (FIG. 20). Each section was then filled with different bio inks (pre-stained cell suspensions) simultaneously. Following 15 minutes of incubation, allowing the cells to settle, the insert was gently removed, enabling cells from both sides to establish cell-cell connections. Intriguingly, the gentle convection fluid flow, during the insert removal, allowed some cells from each side to bridge the gap, resulting in the formation of a well integrated construct with a distinct boundary. After one day of incubation, these sections fused, forming an integrated cell sheet. FIG. 19A-iv illustrates the seamless fusion of DiO and DiL-stained 3T3 cells at the boundary where the two sheets converge. Finally, a free-hand drawing strategy was tested to pattern cells within the cell sheet by sequentially depositing cell suspensions. First, the DiL-tagged 3t3 cells were added to the PDMS mold using a fine pipette tip to draw parallel line patterns, followed by a 15-minute incubation to allow the cells to settle. Then, GFP-3t3 cells were gently added to the PDMS mold to fill out the void spaces without disrupting the previous pattern. After 1 day of incubation at 37° C. and 5% CO₂, a well-integrated cell sheet was created, with the drawn pattern preserved within the sheet which shows the possibility of marbling pattering by adopting this biofabrication technique (FIG. 19A-v1). Another strategy to do marbling pattering was to use a 3D printed external insert with wavy partitions and filling of each section with DiL-stained 3t3 or gfp-3t3 fibroblasts followed by removing the insert after 15 minutes of incubation at 37° C. After one day, a marbled patterned cell sheet formed, and images were taken using the chemidoc imager (FIG. 19A-v2). These methods demonstrate that the rapid formation enable diverse set of ways to pattern multiple cell types in precise locations on these sheets to form heterogenous 3D cell assemblies which are crucial for many applications.

Lack of sufficient vascular networks and lumen structures in thicker tissue engineered constructs (more than 200 μm thickness) cause necrotic core formation within the samples due to the nutrient and oxygen diffusion limitations (Asakawa et al., 2010). Therefore, integration of perfusion channels within these cell sheet constructs can be useful to preserve the functionality of thicker and larger constructs. Human umbilical vein endothelial cells (HUVECs) were seeded into the PDMS mold with high initial cellular density (1.9-2.5×10⁶ cells/cm²), followed by incubation at 37° C. and 5% CO₂ for 2 days. Results indicated that HUVECs do not yield monolithic cell sheet constructs when cultured in the PDMS mold. The HUVECs constructs disaggregated upon removal from the PDMS mold, lacking the robustness required to maintain a contiguous cell sheet after 1 day and formed discrete aggregates. It should be noted that HUVECs cell sheets have been previously made by culturing them on either of temperature-responsive tissue culture plate or nanofiber mesh (M. S. Kim et al., 2017; Sasagawa et al., 2010). Both techniques provide anchorage sites for HUVECs to attach and produce their own ECM components which then can facilitate the integrity of sheets through secretion of ECM materials and stabilization of cell-cell connections. However, using PDMS mold as a non-adherent surface would not provide proper condition for HUVECs to rapidly self-assemble and make robust and well-integrated large area cell sheet. In order to tackle this issue, co-culturing strategies have been developed to utilize the benefits of endothelial cells in engineering vascularized tissue-like constructs.

Studies have shown that endothelial cells are surrounded by ECM components produced by fibroblasts, vascular smooth muscle cells, and pericytes, which are crucial to preserve the function and integrity of endothelial cells in blood vessels (Kusuma et al., 2012). Endothelial cells modulate the ECM components through secreted factors such as transforming growth factor-β (TGFβ) (Kusuma et al., 2012). Therefore, in this study, to prepare the suitable condition for HUVECs to develop prevascularized cell sheets, they have been cocultured with fibroblasts cells. Two co-culturing strategies have been tested to validate the possibility of creating large area self-assembled prevascularized cell sheets. First, a combination of red fluorescent protein-tagged (RFP)-HUVECs and green fluorescent protein-tagged (GFP)-3t3 fibroblast cells, at a 1:4 ratio respectively, were suspended in the fibroblast growth medium and introduced into the 1 cm circular PDMS mold. This co-culture system was incubated at 37° C. and 5% CO₂ for four days, with its microstructural evolution monitored using Zeiss fluorescent microscopy. FIG. 19B shows the formation of microvascular-like network within the cell sheet after four days of coculturing. Without being bound by theory, this suggests that while fibroblast cells play a crucial role to maintain the robustness and integrity of cell sheet, they also produce collagenous ECM for endothelial cells to attach and migrate to form vascular-like network. The second strategy was developed by sequential deposition of HUVECs on top of fibroblast cells. 1.5×10⁶ (GFP)-3t3 fibroblasts were seeded into the 1 cm diameter PDMS mold followed by 2.5 hours of incubation at 37° C. and 5% CO₂. Next, 1.5×10⁶ (RFP)-HUVECs were gently added to the same PDMS mold to prevent integrity disruption of fibroblast layer. After incubation, HUVECs settled down on top of the fibroblast layer and made a bilayer coculture system of prevascularized cell sheet. Fluorescent micrographs of this sample showed the integration of HUVECs to the fibroblasts within 1 day of culture which makes them a prevascularized single unit of self-assembled cell sheet (FIG. 19C). Such self-assembly of HUVECs to develop microvasculature could enhance graft survival after implantation at the injury site by establishing connections with the body's vascular bed. Additionally, it might reduce the likelihood of necrotic core formation in thicker constructs by integrating these perivascular constructs into stacked cell sheet structures (Sekine et al., 2013).

### SUMMARY

A novel biofabrication technique was developed to create planar cell sheet constructs without the use of any external scaffolding materials. This method leverages the inherent ability of adherent cells to self-assemble and form cell-cell connections in the absence of anchoring sites on the culture surface. The technique does not require any specific surface modifications or changes to the cell culture conditions, such as reducing the incubation temperature, altering the culture medium pH, or applying external physical stimuli. Pristine PDMS molds, fabricated in various sizes and shapes, served as low surface energy and low adhesion substrates to encourage cellular self-assembly into cell sheet constructs. The feasibility of this method to fabricate large area cell sheets of diverse morphologies has been demonstrated.

For the creation of thicker and more intricate tissue-like constructs, cell sheets can be layered, assembled to form complex structures, or co-cultured with other cell types using different patterning strategies. Employing such strategies offers the potential to incorporate cell types like HUVECs into the cell sheet, creating perivascular constructs. This integration can reduce the risk of necrotic core formation in thicker constructs, ensuring better integration with host tissue upon implantation. Various in vitro assays were conducted to assess the impact of this technique on cellular behavior, including viability, ECM production, and specific gene expression. Compared to conventional 2D cell cultures, the results indicate that cellular functionality remains largely unaffected within the non-adherent substrate, with minimal impact on cellular viability. A range of cell lines, primary cells, and stem cells including NIH-3T3, 3T3-L1, C2C12, BeWo, Saos-2, human skeletal muscle cells, Burn-Derived Mesenchymal Stem Cells (BD-MSC), and Umbilical Cord-derived Mesenchymal Stem Cells (UC-MSC) were utilized to demonstrate the versatility of this method in creating various tissue models or substitutes.

In an in vivo porcine thermal injury model, it was demonstrated that the UC-MSC cell sheets are biocompatible and reintegrated well into the wound bed.

In contrast to existing methods, this approach introduces a rapid, straightforward, and cost-effective biofabrication technique, eliminating the need for surface treatments or specialized equipment.

While the present disclosure has been described with reference to examples, it is to be understood that the scope of the claims should not be limited by the embodiments set forth in the examples, but should be given the broadest interpretation consistent with the description as a whole.

All publications, patents and patent applications are herein incorporated by reference in their entirety to the same extent as if each individual publication, patent or patent application was specifically and individually indicated to be incorporated by reference in its entirety. Where a term in the present disclosure is found to be defined differently in a document incorporated herein by reference, the definition provided herein is to serve as the definition for the term.

### CITATIONS

Asakawa, N., Shimizu, T., Tsuda, Y., Sekiya, S., Sasagawa, T., Yamato, M., Fukai, F., & Okano, T. (2010). Pre-vascularization of in vitro three-dimensional tissues created by cell sheet engineering. Biomaterials, 31 (14), 3903-3909.
De Pieri, A., Rochev, Y., & Zeugolis, D. I. (2021). Scaffold-free cell-based tissue engineering therapies: advances, shortfalls and forecast. In npj Regenerative Medicine (Vol. 6, Issue 1). Nature Research.
Eylert, G., Dolp, R., Parousis, A., Cheng, R., Auger, C., Holter, M., Lang-Olip, I., Reiner, V., Kamolz, L. P., & Jeschke, M. G. (2021). Skin regeneration is accelerated by a lower dose of multipotent mesenchymal stromal/stem cells—a paradigm change. Stem Cell Research and Therapy, 12 (1).
Kim, M. S., Lee, B., Kim, H. N., Bang, S., Yang, H. S., Kang, S. M., Suh, K.-Y., Park, S.-H., & Jeon, N. L. (2017). 3D tissue formation by stacking detachable cell sheets formed on nanofiber mesh. Biofabrication, 9 (1), 015029.
Kusuma, S., Zhao, S., & Gerecht, S. (2012). The extracellular matrix is a novel attribute of endothelial progenitors and of hypoxic mature endothelial cells. The FASEB Journal, 26 (12), 4925-4936.
Marescal, O., & Cheeseman, I. M. (2020). Cellular Mechanisms and Regulation of Quiescence. In Developmental Cell (Vol. 55, Issue 3, pp. 259-271). Cell Press.
Matsuura, K., Utoh, R., Nagase, K., & Okano, T. (2014). Cell sheet approach for tissue engineering and regenerative medicine. In Journal of Controlled Release (Vol. 190, pp. 228-239). Elsevier B. V.
Murray, L. A., Knight, D. A., & Laurent, G. J. (2009). Fibroblasts. In Asthma and COPD (pp. 193-200). Elsevier.
Nozaki, T., Yamato, M., Inuma, T., Nishida, K., & Okano, T. (2008). Transportation of transplantable cell sheets fabricated with temperature-responsive culture surfaces for regenerative medicine. Journal of Tissue Engineering and Regenerative Medicine, 2 (4), 190-195.
Qian, Z., Ross, D., Jia, W., Xing, Q., & Zhao, F. (2018). Bioactive polydimethylsiloxane surface for optimal human mesenchymal stem cell sheet culture. Bioactive Materials, 3 (2), 167-173.
Sasagawa, T., Shimizu, T., Sekiya, S., Haraguchi, Y., Yamato, M., Sawa, Y., & Okano, T. (2010). Design of prevascularized three-dimensional cell-dense tissues using a cell sheet stacking manipulation technology. Biomaterials, 31 (7), 1646-1654.
Sekine, H., Shimizu, T., Sakaguchi, K., Dobashi, I., Wada, M., Yamato, M., Kobayashi, E., Umezu, M., & Okano, T. (2013). In vitro fabrication of functional three-dimensional tissues with perfusable blood vessels. Nature Communications, 4.
Shahin-Shamsabadi, A., & Selvaganapathy, P. R. (2020). π-SACS: PH Induced Self-Assembled Cell Sheets without the Need for Modified Surfaces. ACS Biomaterials Science and Engineering, 6 (9), 5346-5356.
Takezawa, T., Mori, Y., Yonaha, T., & Yoshizato, K. (1993). Characterization of Morphology and Cellular Metabolism during the Spheroid Formation by Fibroblasts. Experimental Cell Research, 208 (2), 430-441.
Takezawa, T., Mori, Y., & Yoshizato, K. (1990). Cell Culture on a Thermo-Responsive Polymer Surface. Nature Biotechnology, 8 (9), 854-856.

1. A method of forming a cell sheet, comprising:
seeding cells with a culture medium on to a substantially flat substrate, the substrate having low surface energy and/or ultralow adhesion; and
incubating cells on the substrate under time and temperature conditions to form a cell sheet;
wherein the cells are seeded on the substrate to a monolayer density of at least 65%.

2. The method of claim 1, wherein the cells are seeded on the substrate to a monolayer density of 90 to 100%.

3. The method of claim 1, wherein the incubation time ranges from 20 minutes to 10 hours.

4. The method of claim 1, wherein the incubation temperature ranges from 25° C. to 39° C.

5. The method of claim 1, wherein the cell sheet is a 3-dimensional (3D) cell sheet.

6. The method of claim 1, wherein seeding the cells comprises providing the cells to a mold, the bottom of the mold comprising the substrate, optionally wherein the mold defines the shape of the cell sheet.

7. The method of claim 1, wherein the substantially flat substrate comprises at least one topographical feature.

8. The method of claim 1, wherein the substrate is selected from the group consisting of a silicone polymer, a fluoropolymer, a passivated or native polyolefin, a denser than water liquid, or a combination thereof.

9. The method of claim 8, wherein the silicone polymer comprises polydimethylsiloxane (PDMS), optionally pristine PDMS.

10. The method of claim 1, wherein the cells are selected from human cells or animal cells.

11. The method of claim 1, wherein two or more types of cells are seeded and incubated at different areas of the substrate.

12. The method of claim 11, wherein the two or more types of cells are seeded and incubated at different areas divided by an insert on the substrate.

13. The method of claim 12, wherein the insert has a thickness that ranges from 0.1 mm to 10 mm, optionally less than 4 mm.

14. The method of claim 12, wherein the insert is used to segment the substrate area for selective seeding of cells and is removed within 10 minutes to 1 hour of cell seeding.

15. The method of claim 1, wherein the culture medium is selected from the group consisting of a biological media, optionally plasma, serum, or diluted tissue extract, a synthetic media, optionally DMEM, EBM, EBM-2, F10, or F12, or a combination thereof.

16. A method of constructing a multi-layer cell sheet, comprising: stacking two or more of the cell sheets produced by the method of claim 1 in a culture medium, wherein the cell sheets mutually fuse under time and temperature conditions.

17. The method of claim 16, wherein the time ranges from 10 minutes to 10 hours and/or wherein the temperature ranges from 25° C. to 39° C.

18. A method of constructing a two-dimensional (2D) or a three-dimensional (3D) cell construct comprising: overlapping the edges of two or more of the cell sheets produced by the method of claim 1 in a culture medium, wherein the edges mutually fuse under time and temperature conditions.

19. The method of claim 18, wherein the time ranges from 10 minutes to 10 hours and/or wherein the temperature ranges from 25° C. to 39° C.

20. A cell sheet made according to the method of claim 1, wherein the cell sheet is an artificial meat product or is for wound healing, regenerative medicine or drug discovery applications.
